(19) United States
(12) Patent Application Publication
(10) Pub. No.: US 2016/0015777 A1
(43) Pub. Date: Jan. 21, 2016

(54) Protein Delivery System and Method of Making Same

(71) Applicant: Nutraceutical Strategies Corporation, Whiteville, NC (US)

(72) Inventor: Daniel Anthony Traylor, Whiteville, NC (US)

(73) Assignee: Nutraceutical Strategies Corporation, Whiteville, NC (US)

(21) Appl. No.: 14/800,775

(22) Filed: Jul. 16, 2015

Related U.S. Application Data
(60) Provisional application No. 62/025,298, filed on Jul. 16, 2014.

(51) Int. Cl. A61K 38/17; A61K 9/00; A23G 3/36; A61K 31/05; A61K 38/43; A23G 3/44; A61K 31/198; A61K 47/42

(52) U.S. Cl. CPC A61K 38/17; A61K 31/198; A61K 9/0056; A61K 47/42; A61K 31/05; A61K 38/43; A23G 3/44; A23G 3/368; A23V 2002/00

(57) ABSTRACT

A protein delivery system that may provide a complete essential amino acid status, stimulating muscle growth and maintaining muscle mass, while reducing caloric intake.

### BACKGROUND OF THE INVENTION

1) Field of the Invention

The present invention relates to a protein delivery system and a method of making same. The system may provide a complete essential amino acid status and may stimulate muscle growth and maintain muscle mass, while reducing caloric intake and promoting overall health. The system may also combat potential health threats.

2) Description of Related Art

Consumers are becoming more quality conscious regarding the use and content of nutritional products. However, many nutritional products, particularly protein supplements, do not follow scientific principles when producing their respective formulations. For example, organizations will market protein supplements containing amino acid complexes that do not stimulate muscle growth or muscle maintenance, yet claim that this is a possible result of using their supplement. This confuses health conscious consumers and misleads more. Thus, it is apparent there is a technological gap in the formulation of effective protein supplements.

Moreover, malnutrition is still a health risk for many intensive/non-intensive cares patients, which describes as a broad population of patients who may suffer from a variety of diseases or insults, such as diabetes, who are fed either with parenteral formulations or enteral formulations either to replace or supplement a typical diet. It was reported in 1991 and 2014 that on average 13% and 43% of hospitalized patients had a prevalence of malnutrition that required nutritional support beyond food. Of these patients, at least 62% were supported using similar formulations with 30% from oral supplements (Gray et al., U.S. Pat. No. 5,714,472, Feb. 3, 1998; Lazarus C, Hamlyn J. Prevalence and documentation of malnutrition in hospitals: a case study in a large private hospital setting. Nutr Diet. 2005; 62(1):41-4710; Pirlich M, Schutz T, Kemps M, et al. Social risk factors for hospital malnutrition. Nutrition. 2005; 21(3):295-300).

Yet in another study, it was reported that 40% of patients lost more than 10 kilograms (22.2 pounds) of body weight during and at follow-up directly after ICU admission (Kvale R, Ulvik A, Flaatten H. Follow-up after intensive care: a single center study. Intensive Care Med. 2003; 29(12):2149-2156). It is possible that significant decreases in muscle mass (i.e., sarcopenia) contributed to losses in body weight. It is apparent that elderly patients, with malignant disease(s), and patients receiving four or more medications (i.e., polypharmacy) are also at increased risk for the development of malnutrition, while hospitalized (Pirlich M, Schutz T, Kemps M, et al. Social risk factors for hospital malnutrition. Nutrition. 2005; 21(3):295-300). Additionally, athletes and health conscious individuals may exhibit metabolic stresses from exercise, particularly resistance and endurance training, and thus may require similar nutritional support.

One important aspect of nutritional compounds is the use of proteins. Proteins are large biological molecules, or macromolecules, consisting of one or more long chains of amino acid residues. Proteins perform a vast array of functions within living organisms, including catalyzing metabolic reactions, replicating DNA, responding to stimuli, and transporting molecules from one location to another. Proteins differ from one another primarily in their sequence of amino acids, which is dictated by the nucleotide sequence of their genes. The sequence usually results in folding of the protein into a specific three-dimensional structure that determines its activity.

A linear chain of amino acid residues is called a polypeptide. A protein contains at least one long polypeptide. Short polypeptides, containing less than about 20-30 residues, are rarely considered to be proteins and are commonly called peptides, or sometimes oligopeptides. The individual amino acid residues are bonded together by peptide bonds and adjacent amino acid residues.

Shortly after, or even during, synthesis, the residues in a protein are often chemically modified by post-translational modification, which alters the physical and chemical properties, folding, stability, activity, and ultimately, the function of the proteins. Sometimes proteins have non-peptide groups attached, which can be called prosthetic groups or cofactors. Proteins can also work together to achieve a particular function, and they often associate to form stable protein complexes.

Once formed, proteins only exist for a certain period of time and are then degraded and recycled by the cell's machinery through the process of protein turnover. A protein's lifespan is measured in terms of its half-life and covers a wide range. Proteins can exist for minutes or years with an average lifespan of 1-2 days in mammalian cells. Abnormal and or misfolded proteins are degraded more rapidly either due to being targeted for destruction or being unstable.

Like other biological macromolecules, such as polysaccharides and nucleic acids, proteins are essential parts of organisms and participate in virtually every process within cells. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signaling, immune responses, cell adhesion, and the cell cycle. Proteins are also necessary in animals' diets, since animals cannot synthesize all the amino acids they need and must obtain essential amino acids from food. Through the process of digestion, animals break down ingested protein into free amino acids that are then used in metabolism.

Most microorganisms and plants can biosynthesize all 20 standard amino acids, while animals (including humans) must obtain some of the amino acids from their diet. The amino acids that an organism cannot synthesize on its own are referred to as essential amino acids. Key enzymes that synthesize certain amino acids are not present in animals—such as aspartokinase, which catalyzes the first step in the synthesis of lysine, methionine, and threonine from aspartate.

Ingested proteins are broken down into amino acids through digestion, which typically involves denaturation of the protein through exposure to acid and hydrolysis by enzymes called proteases. Some ingested amino acids are used for protein biosynthesis, while others are converted to glucose through gluconeogenesis, or fed into the citric acid cycle. This use of protein as a fuel is particularly important under starvation conditions as it allows the body's own proteins to be used to support life, particularly those found in muscle. Amino acids are also an important dietary source of nitrogen

Proteins are essential nutrients for the human body. They are one of the building blocks of body tissue, and can also serve as a fuel source. For example, skeletal muscle comprises about 43% of body mass and uses a disproportionate amount of the body's energy reserves during exercise, however, studies have suggested that many individuals (e.g., elderly women) do not consume enough daily protein.

Whether high-protein diets are detrimental to health is controversial, however, studies have shown that individuals (e.g., weight lifters) who consumed up to 3 grams of protein/kilogram of body weight exhibited no adverse effects. It has also been reported that extremely high doses of protein (300 grams/day) are not uncommon intakes. As a fuel, proteins contain 4 kcal per gram, just like carbohydrates and unlike lipids, which contain 9 kcal per gram. During human digestion, proteins are broken down in the stomach and small intestine to smaller polypeptide chains via hydrochloric acid and protease actions. This is crucial for the synthesis of the essential amino acids that cannot be biosynthesized by the body.

Different proteins have different levels of biological availability to the human body. Many methods have been introduced to measure protein utilization and retention rates in humans. They include biological value, net protein utilization, and PDCAAS (Protein Digestibility Corrected Amino Acids Score), which was developed by the FDA as an improvement over the Protein efficiency ratio (PER) method. These methods examine which proteins are most efficiently used by the body. The PDCAAS rating is a fairly recent evaluation method; it was adopted by the US Food and Drug Administration (FDA) and the Food and Agricultural Organization of the United Nations/World Health Organization (FAO/WHO) in 1993 as “the preferred ‘best’” method to determine protein quality. These organizations have suggested that other methods for evaluating the quality of protein are inferior.

Amino acids can be divided into three categories: essential amino acids, non-essential amino acids, and conditional amino acids. As discussed, Essential amino acids cannot be made by the body, and must be supplied by food. Non-essential amino acids are made by the body from essential amino acids or in the normal breakdown of proteins. Conditional amino acids are usually not essential, except in times of illness, stress, or for someone challenged with a lifelong medical condition. Essential amino acids include leucine, isoleucine, valine, lysine, threonine, tryptophan, methionine, phenylalanine, and histidine. Non-essential amino acids include alanine, asparagine, aspartic acid, and glutamic acid. Conditional amino acids include arginine, cystine, glutamine, glycine, proline, serine, and tyrosine. The current disclosure is considered to encompass these amino acids as well as salts or derivatives thereof.

The foods richest in essential amino acids are those from animal sources such as meats, dairy products, fish and eggs. Although lower in essential amino acids, plant sources also contain protein: whole grains, pulses, legumes, soy, fruits, nuts and seeds. Vegetarians and vegans may obtain essential amino acids by eating a variety of plant proteins. Amino acids, the building blocks of proteins, are used for building muscle tissue and repairing damaged tissues.

Given the importance of proteins and their constituent amino acids to the human body, various studies have been conducted with respect to the effects of proteins in association with exercise. For example, An Oral Essential Amino Acid-Carbohydrate Supplement Enhances Muscle Protein Anabolism After Resistance Exercise, Erin L. Glynn, et al., 2010 http://ajpregu.physiology.org/content/299/2/R533.short.

The study used the following experimental design:

35 g sucrose+6 g EAA L-Phenylalanine 0.93 g

L-Valine 0.7 g

L-Threonine 0.88 g

L-Isoleucine 0.6 g

L-Methionine 0.19 g

L-Histidine* (essential in infants) 0.65 g

L-Leucine 1.12 g

L-Lysine 0.93 g

*0.0605 g of L-[ring-2H5]phenylalanine was added to maintain steady state equilibrium

The results showed insulin concentrations elevated, with a peak between 20 and 30 minutes after consumption for the groups participating in the study. Muscle protein synthesis was significantly increased but there was no difference in protein synthesis between protein drinks administered between the 1 and 3 hour marks of the experiment. Muscle protein break down did not change. The study determined exercise followed by ingestion of essential amino acids increases muscle protein synthesis in humans and increases signal transduction of rapamycin complex 1 (mTORC1). In addition, low carbohydrate and high carbohydrate values were equal in causing muscle protein synthesis, and decreasing muscle breakdown consistent that post-exercise muscle protein synthesis is not enhanced with excess (>40 g) carbohydrate.

A further study, Timing Of Amino Acid-Carbohydrate Ingestion Alters Anabolic Response Of Muscle To Resistance Exercise, Kevin D. Tipton et al., 2001, http://ajpendo.physiology.org/content/281/2/E197 used the following experimental design:

5 grams of sucrose in 500 ml H₂O

L-Histidine 0.65 mg, 4.2 μmol

L-Isoleucine 0.60 mg, 4.6 μmol

L-Leucine 1.12 mg, 8.5 μmol

L-Lysine 0.93 mg, 6.4 μmol

L-Methionine 0.19 mg, 1.3 μmol

L-Phenylalanine 0.93 mg, 5.6 μmol

L-Threonine 0.88 mg, 7.4 μmol

L-Valine 0.7 mg, 6.0 μmol

*0.0605 grams of-[ring-2H5]phenylalanine and aspartame

The results indicated that delivery of essential amino acids was significantly greater in PRE than in POST during the exercise bout and in the first hour after exercise. Total net phenylalanine uptake across the leg was greater during PRE (209±42 mg) than during POST (81±19). Phenylalanine disappearance rate, an indicator of MPS from blood amino acids, increased after essential amino acid-carbohydrate consumption (EAC) in both trials. Thus, indicating that net MPS response to the consumption of an EAC solution immediately before resistance exercise is greater than that when the solution is consumed after exercise. This is likely due to an increased delivery rate of amino acids to the muscle(s). The results also indicated that oral or intravenous supplementation of essential amino acid supplementation combined with table sugar (i.e., sucrose) promotes high insulin spikes when consumed before or after exercise.

A further study, Independent and combined effects of liquid carbohydrate/essential amino acid ingestion on hormonal and muscular adaptations following resistance training in untrained men, Bird S P, et al., 2006 http:/www.ncbi.nlm.nih.gov/pubmed/16456674 used the following experimental design

32 untrained males supplemented for six months

### Experimental Design

5 grams of sucrose in 500 ml H₂O

L-Histidine 0.65 grams

L-Isoleucine 0.6 grams

L-Leucine 1.12 grams

L-Lysine 0.93 grams

L-Methionine 0.19 grams

L-Phenylalanine 0.93 grams

L-Threoine 0.88 grams

L-Valine 0.7 grams

The results indicated that muscle cross-sectional area (mCSA) increased for type I, IIa, and IIb muscle fibers with increases in post exercise blood levels. Supplementation with low levels of carbohydrate plus essential amino acids displayed the greatest gains in mCSA. When compared to essential amino acids, MPS was greater for the group that consumed carbohydrate combined with essential amino acids. These data also indicated that MPS following resistance training produced the highest MPS levels. Thus, consumption of essential amino acids stimulates MPS independent of sucrose (i.e., table sugar) and resistance training, while attenuating protein degradation. Resistance training, however, has the greatest effects on stimulating MPS. The authors (Bird et al., 2006) suggested that 15 grams of essential amino acids stimulates muscle protein synthesis and maintains net protein balance.

A further study, Supplementation of a suboptimal protein dose with leucine or essential amino acids: effects on myofibrillar protein synthesis at rest and following resistance exercise in men, Churchward-Venne T A, et al., 2012 DOI: 10.1113/jphysio1.2012.228833 used the following experimental design:

8 recreationally active males supplemented with whey protein combined with L-leucine

### Experimental Design

6.25 grams of whey protein isolate supplemented with free-form leucine (total leucine=3.0 grams) were administered, in a single-blinded manner, with 300.0 mL H2O.

The results indicated leucinaemia was equivalent to absorbing 25.0 grams of whey protein isolate. During a fasted condition, muscle protein synthesis (MPS) also increased from one to three hours prior to and following a bout of exercise. From three to five hours post ingestion MPS remained 55% above baseline levels. Additionally, amino acid transporter mRNA abundance increased above the fasting condition and after exercise. Thus, the authors concluded that a low dose of whey protein supplemented with L-leucine was as effective as consuming a complete whey protein source for promoting MPS.

The current disclosure is set apart from the previously mentioned studies due to the combination of whole protein and sufficient amounts of amino acids and phenols that may be infused into novel delivery systems to overcome the shortfalls of the above disclosures. Accordingly, it is an object of the present disclosure to provide scientifically based and designed protein sources or supplements that overcome the various failings, and false claims, present in many currently available nutritional supplements. It is also an object of this disclosure that protein supplement requires no preparation before eating and leaves behind no container or packaging materials once consumed. Another object of the disclosure is to provide a protein supplement that tastes like candy. Thus, a user may satisfy their sweet tooth while ingesting protein, which will keep the user feeling full longer.

### SUMMARY OF THE INVENTION

The above objectives are accomplished according to the present invention by providing a protein delivery system comprising at least one protein, at least one essential amino acid, at least one caloric intake reducing agent, at least one anti-carcinogenic factor; and wherein the protein delivery system is a semi-solid. In one embodiment, A protein delivery system is disclosed. The system may include at least one protein, at least one essential amino acid, at least one caloric intake reducing agent, at least one anti-carcinogenic factor, and the protein delivery system may be a semi-solid.

In a further embodiment, the protein delivery system may include the at least one protein from 14 to 20 percent by weight. Further the at least one essential amino acid may be present from 3 to 9 percent by weight. In another embodiment, the at least one anti-carcinogenic factor may be present from 0.2 to 0.8 percent by weight.

In an alternative embodiment, a protein delivery system is disclosed that may include at least one protein source, at least one amino acid source, at least one amino acid metabolite source, at least one enzyme source, at least one lipid source, at least one vitamin, at least one mineral, at least one botanical/herbal-extract, at least one caloric intake reducing agent, at least one anti-carcinogenic factor, the protein delivery system may be a semi-solid.

In a further embodiment, the protein source may be present from 14 to 20 percent by weight. In another embodiment, the amino acid source may be present from 3 to 9 percent by weight. In a further embodiment, the lipid source may be present from 3 to 9 percent by weight. Still further, the lipid source may include omega 3 and omega 6 fatty acids. Even further, the anti-carcinogenic factor may be present from 0.2 to 0.8 percent by weight. Even yet further, the vitamin source may be present from 0.2 to 0.8 percent by weight.

In another alternative embodiment, a method of forming a gummy protein delivery device is disclosed. The method may include forming a gummy matrix by integrating ingredients, heating the gummy matrix, adding gelatin to the gummy matrix and mixing the gummy matrix until the gelatin is completely dissolved, maintaining the resulting gummy matrix at a constant temperature, slow boiling the gummy matrix, adding a sweetener to the gummy matrix, incorporating ergogenic ingredients into the gummy matrix, homogenously mixing the gummy matrix, and molding the resulting gummy matrix.

In another embodiment, the gummy matrix may be a mixed gel. In a further embodiment, the mixed gel may comprise from 7 to 9 percent by weight gelatin. Still further, sugar may be added to the gummy matrix from 5.0 to 8.0 percent by weight of the gummy matrix. In another embodiment, a protein source is added to the gummy matrix. Still further, the protein source may comprise from 14.0 to 22.0 percent by weight of the gummy matrix. Still further, gelatin is not added to the gummy matrix until the matrix is at a temperature of from 55 to 70 degrees Celsius. In another embodiment, ergogenic ingredients are only added after the gummy matrix has cooled to a temperature within the range of 40 to 45 degrees Celsius.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a table of amino acids and content for same derived from larval silkworms.

FIG. 2 shows the protein characteristics and applications of various protein.

FIG. 3 shows a general schematic diagram of micro/nano-particles with certain bioactive proteins, peptides, essential/non-essential amino acids, amino acid metabolites, regulatory nutrients, and drugs.

FIG. 4 shows the net charge of Alanine based upon the pH of the surrounding aqueous environment during the titration.

FIG. 5, shows the effects of different concentrations of Trans-Resveratrol on low density lipoprotein oxidation

FIG. 6 shows a graph of arterialized blood temperature measures.

FIG. 7 shows a graph of leucine blood measures.

FIG. 8 shows a graph of branched chain amino acid blood measures.

FIG. 9 shows a graph of essential amino acid blood measures.

FIG. 10 shows a graph of non-essential amino acid blood measures.

FIG. 11 shows a graph of conditionally essential amino acid blood measures.

FIG. 12 shows a graph of glucose blood measures.

FIG. 13 shows a graph of insulin blood measures.

FIG. 14 shows the mean (±standard error of the mean [SEM]) values for baseline 1, baseline 2, and all time-points for arterialized blood temperatures and blood concentrations of leucine, branched chain amino acids, essential amino acids, non-essential amino acids, conditional amino acids, glucose, and insulin.

FIG. 15 shows the mean (±standard error of the mean [SEM]) values for baseline 1, baseline 2, and all time-points for arterialized blood temperatures and blood concentrations of leucine, branched chain amino acids, essential amino acids, non-essential amino acids, conditional amino acids, glucose, and insulin.

FIG. 16 shows arterialized blood values and blood concentrations (mean±SEM) of leucine, branched chain amino acids (BCAA), essential amino acids (EAA), non-essential amino acids (NEAA), conditional amino acids (CEAA), glucose, and insulin at pre-ingestion 1 (0955-0959 EST), pre-ingestion 2 (1034 EST), and at each post-ingestion time-point (1114-1340 EST).

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

The current disclosure relates to a protein delivery system and a method of making same. The system may provide a bioactive protein, peptide, and essential/non-essential amino acid, metabolite, drug, and other macronutrient and regulatory nutrient status, while being used in conjunction with disease/mental-health cure and prevention treatment protocols. Additionally, the system may also stimulate muscle growth, maintain muscle mass, improve absorption capacity and nutrient/drug-composition delivery, while reducing caloric intake, supporting dietary reference nutrition, promoting overall health, and enhancing physical well-being. The system may also combat potential health treats and supplement the unique nutrient needs of individuals that are generated due to exercise, tissue repair, and healing requirements. Furthermore, a method of making same may improve the integrity and effectiveness of the invention.

The current disclosure may also combat potential health threats and have applications in clinical settings for clinicians who develop nutritional or drug administration protocols to help patients' return to work and athletes' return to practice and competition. For example, solid colloidal (semi-solid) nano-particles used to entrap, encapsulate or absorb active protein bound agents (Azimi et al. Producing gelatin nanoparticles as delivery system for bovine serum albumin. Iranian Biomedical Journal. 2014; 18(1):34-40; DOI: 10.6091/ibj.1242.2013) may be used in the present inventions parenteral or enteral formulations, through numerous natural and synthetic materials, as well as, processing and manufacturing methods.

In one embodiment, the present disclosures provides an improved protein source. Proteins are believed to increase performance in terms of athletics as well as have other beneficial health impacts. The type of protein provided is important in terms of its influence on protein metabolic response and on muscle exercise performance. The different physical and/or chemical properties within the various types of proteins may affect the rate of protein digestion. As a result, the amino acid availability and the accumulation of tissue protein is altered because of the various protein metabolic responses.

Various sources may be used for processing, manufacturing, and providing protein for the present disclosure. These various sources yield a multitude of proteins for use in the protein source disclosed herein. For instance, whey protein is a dairy-based source of amino acids and is believed to improve athletic performance, particularly in resistance training. Whey is conducive to muscle growth and maintenance. When combined with a resistance training program, whey protein is believed to increase body cell mass, muscle mass and muscle strength. For example, it is believed that whey protein may produce improvement in knee extension peak torque (strength) and lean tissue mass. Whey protein may also increase immune response, an advantage for those who tax their immune systems with strenuous, long-duration exercise.

Another protein, casein, is a tasteless, odorless protein derived from milk. It is believed to increase strength during resistance training. It is believed casein proteins produce a greater average increase in upper and lower body strength. It is also believed there is an association between greater strength increases in casein-supplemented users with improved nitrogen retention and overall anti-catabolic effects related to the peptide components of the casein protein.

Other possible protein resources include soy proteins. As exercise and daily living activities cause oxidative damage to muscle, soy protein is believed to have antioxidative properties that provide additional benefits to individuals beyond other sources of supplemental protein. Soy protein contains essential amino acids like other proteins, however, unlike many other protein sources, it is understood to provide cholesterol lowering (i.e., “bad” cholesterol equates with low density lipoprotein and very low density lipoprotein) and cancer-fighting benefits. Soy may actually help increase lean body mass due to its anabolic properties. It is believed that increases in lean body mass occur when administered daily servings of micronutrient-fortified soy protein bars formulated with soy protein or an equivalent amount of whey protein. It is also believed that both soy and whey proteins promote exercise-induced lean body mass gain, but soy protein is believed to produce the added benefit of preserving antioxidant function for decreasing oxidative stress to the body's cells.

A high-quality protein is egg protein. Eggs offer functional nutrients not found to the same extent in dairy proteins or meats. Also, egg protein may have beneficial effects. For example, it is believed that consuming 20 grams of egg protein post-workout may induce peak protein synthesis.

Beef proteins, derived from cattle meat, are a rich source of nutrients, such as creatine, that support muscle gains. It is believed creatine improves muscular endurance in contractile proteins found in skeletal muscle. It is hypothesized that beef has an advantage as a protein reservoir due to its culinary versatility and attractiveness to many strength trainers.

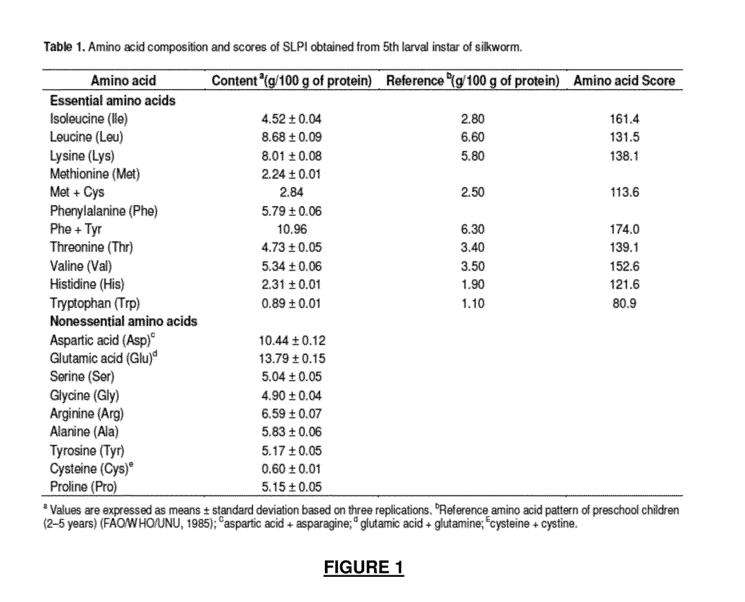

Insect protein has high rates of digestibility in humans, and therefore, may be a good source for supplementation. Silkworm protein is an important protein source, which is consumed in China, Japan, Korea, India and Thailand. Recent studies have reported that silkworm pupae contain 45 to 55% protein (18 amino acids, including 8 essential amino acids) on a dry matter basis. Only recently has information about the nutritional quality and physicochemical properties of silkworm larvae protein isolate. (Wu et al. (2011). Physicochemical properties of silkworm larvae protein isolate and gastrointestinal hydrolysate bioactivities. African Journal of Biotechnology. 10(32): 6145-6153. ISSN 1684-5315; Raksakantong P et al. (2010). Fatty acids and proximate composition of eight Thai edible terricolous insects. Food Res. Int. 43(1): 350-355; Zhou J, Han D (2006). Safety evaluation of protein of silkworm (Antheraea pernyi) pupae. Food Chem. Toxicol. 44(7): 1123-1130. FIG. 1 shows a table of amino acids and content for same derived from larval silkworms.

Proteins may be purified from other cellular components using a variety of techniques such as ultracentrifugation, precipitation, electrophoresis, and chromatography; the advent of genetic engineering has made possible a number of methods to facilitate purification. Methods commonly used to study protein structure and function include immunohistochemistry, site-directed mutagenesis, X-ray crystallography, nuclear magnetic resonance and mass spectrometry.

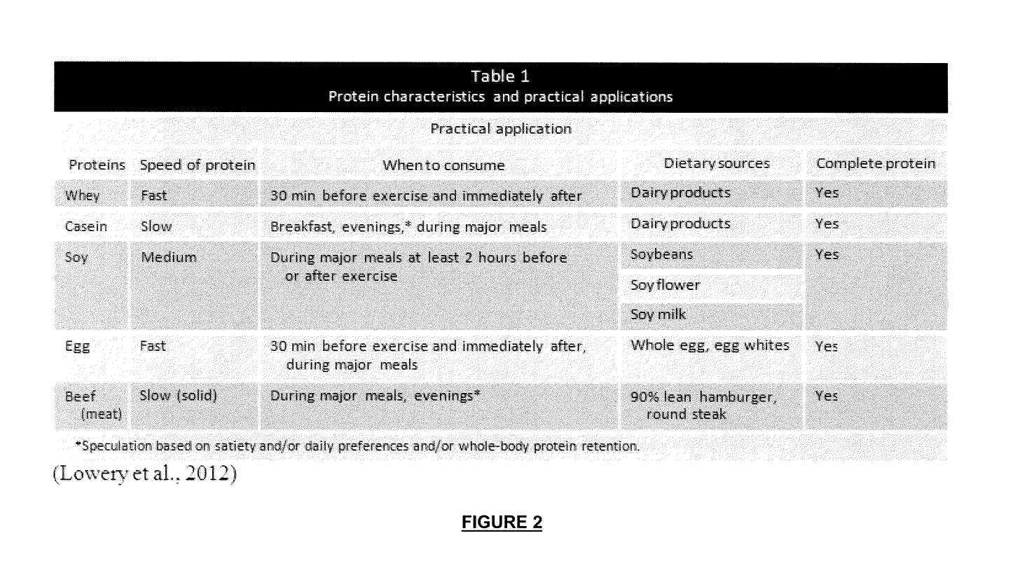

FIG. 2 shows the protein characteristics and applications of various protein.

Table 1, shown below, shows a description and chemical nature of selected amino acids.

TABLE 1

| | | | | | |
|---|---|---|---|---|---|
| | | | pK₁ | pK₂ | pK R |
| Amino Acid | Symbol | Structure* | (COOH) | (NH₂) | Group |

Amino Acids with Aliphatic R-Groups

| | | | | |
|---|---|---|---|---|
| Glycine | Gly-G | 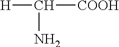 | 2.4 | 9.8 |
| Alanine | Ala-A | 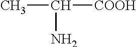 | 2.4 | 9.9 |
| Valine | Val-V | 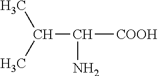 | 2.2 | 9.7 |
| Leucine | Leu-L | 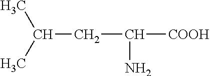 | 2.3 | 9.7 |
| Isoleucine | Ile-I | 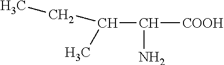 | 2.3 | 9.8 |

Non-Aromatic Amino Acids with Hydroxyl R-Groups

| | | | | | |
|---|---|---|---|---|---|
| Serine | Ser-S | 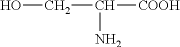 | 2.2 | 9.2 | ~13 |
| Threonine | Thr-T | 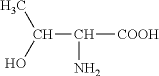 | 2.1 | 9.1 | ~13 |

Amino Acids with Sulfur-Containing R-Groups

| | | | | | |
|---|---|---|---|---|---|
| Cysteine | Cys-C | 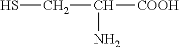 | 1.9 | 10.8 | 8.3 |
| Methionine | Met-M | 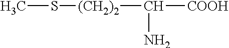 | 2.1 | 9.3 | |

Acidic Amino Acids and their Amides

| | | | | | |
|---|---|---|---|---|---|
| Aspartic Acid | Asp-D | 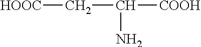 | 2.0 | 9.9 | 3.9 |
| Asparagine | Asn-N | 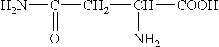 | 2.1 | 8.8 | |
| Glutamic Acid | Glu-E | 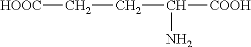 | 2.1 | 9.5 | 4.1 |
| Glutamine | Gln-Q | 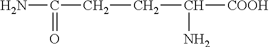 | 2.2 | 9.1 | |

Basic Amino Acids

| | | | | | |
|---|---|---|---|---|---|
| Arginine | Arg-R | 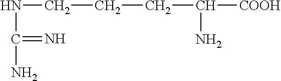 [SMILES: N=C(N)NCCCC(N)C(=O)O] | 1.8 | 9.0 | 12.5 |
| Lysine | Lys-K | 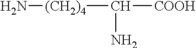 | 2.2 | 9.2 | 10.8 |
| Histidine | His-H | 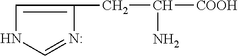 | 1.8 | 9.2 | 6.0 |

Amino Acids with Aromatic Rings

| | | | | | |
|---|---|---|---|---|---|
| Phenylalanine | Phe-F | 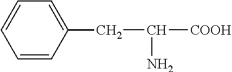 [SMILES: NC(Cc1ccccc1)C(=O)O] | 2.2 | 9.2 | |
| Tryosine | Try-Y | 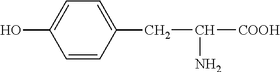 [SMILES: NC(Cc1ccc(O)cc1)C(=O)O] | 2.2 | 9.1 | 10.1 |
| Tryptophan | Trp-W | 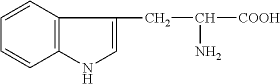 [SMILES: NC(Cc1c[nH]c2ccccc12)C(=O)O] | 2.4 | 9.4 | |

Imino Acids

| | | | | |
|---|---|---|---|---|
| Proline | Pro-P | 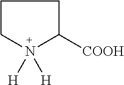 | 2.0 | 10.6 |

R—COOH<-------->R—COO⁻+H⁺

R—NH₃⁺<-------->R—NH₂+H⁺

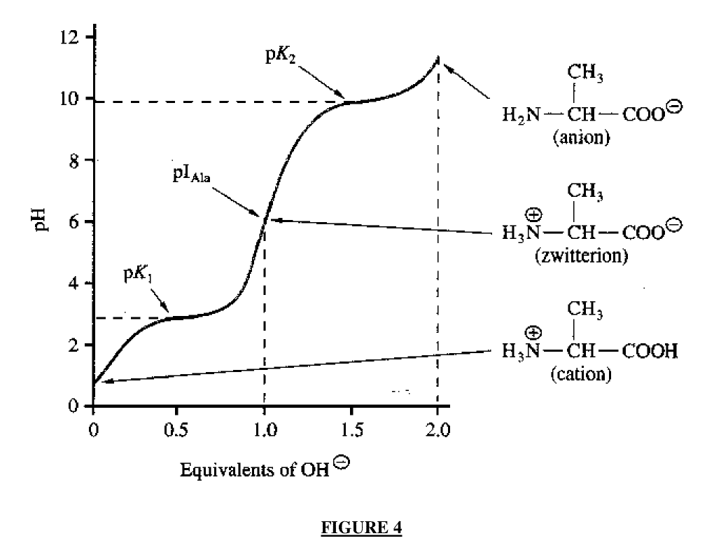

FIG. 4 shows the net charge of Alanine based upon the pH of the surrounding aqueous environment during the titration.

In a further embodiment, the protein source of the present disclosure may include branched chain amino acids. A branched-chain amino acid (BCAA) is an amino acid having aliphatic side-chains with a branch, a carbon atom bound to more than two other carbon atoms. Among the proteinogenic amino acids, there are three BCAAs: leucine, isoleucine and valine.

BCAAs are among the nine essential amino acids for humans, accounting for 35% of the essential amino acids in muscle proteins and 40% of the preformed amino acids required by mammals. BCAAs comprise L-Leucine, L-Isoleucine, and L-Valine. The chemical structure of the three BCAAs is illustrated below:

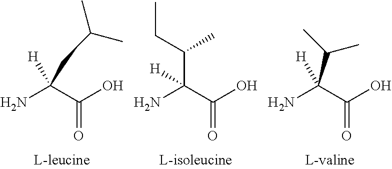

L-leucine may: (1) be oxidized by skeletal muscle for energy, (2) enhance signal protein synthesis, primarily through the anabolic effects of L-Leucine, (3) reduce exercise-induced muscle damage, may prevent fatigue, enhance time to exhaustion, and boost mental acuity by maintaining the ‘BCAA pool’ in the body and preventing the synthesis of serotonin in the brain, and (4) may enhance growth hormone and help prevent decreases in testosterone during and after exercise, promoting anabolism. L-Leucine increases lean muscle mass more rapidly than supplementing with other amino acids. This is due to decreases in muscle wasting and muscle soreness after strenuous activities. L-leucine may also improve immune system function and improve stamina better than supplementing with solely whole proteins. Further, L-Leucine enriched proteins allow these positive effects to occur in the absence of sugar. Use of L-Leucine may also reduce the amount of other proteins needed in the composition.

L-Leucine is believed to either directly or indirectly stimulate muscle protein synthesis or decrease muscle protein degradation via the metabolites alphaketoisocaproate (KIC) acid and beta-hydroxy-beta-methylbutyrate (HMB). In addition, L-Leucine consumption promotes insulin secretion, which can also reduce muscle catabolism after exercise. L-Leucine can stimulate protein translation via the mTOR in part through AMPK activation, or independently, in human cells. Skeletal muscle protein synthesis and eIF4E eIF4G assembly are enhanced by the administration of L-Leucine in the presence of rapamycin (inhibitor of mTOR). Additionally, oral L-Leucine administration may enhance the phosphorylation of 4E-BP1, showing the stimulation effect of L-Leucine on protein synthesis in skeletal muscle. Oral administration of L-Leucine-rich may increase protein synthesis in skeletal muscle. L-Leucine impacts S6K1 activity, which is associated with increased protein synthesis and enhanced translation of mRNA, may have activated eIF factors and/or the S6 kinase pathway. L-Leucine may also increase the expression of eIF4G in skeletal muscle. Increase in the amount of eIF4G may result in greater binding to eIF4E and initiation, which depends on the availability of eIF4E.

Branched chain amino acids may also reduce exercise-induced muscle damage, fight fatigue, enhance time to exhaustion, and boost mental acuity, while enhancing growth hormone production. In addition, BCAAs may help prevent decreases in testosterone during and after exercise, promoting muscle anabolism or muscle building and maintenance.

In one embodiment, Creatine, an amino acid derivative found at high concentrations within skeletal muscles, commonly used as an ergogenic aid may be included in the composition. Creatine, such as for purposes of example and not intended to be limiting, creatine monohydrate, creatine HCL, and PEG-creatine, is believed to be involved in the phosphocreatine metabolic pathway and is believed to increase strength, power, and endurance. Specifically, polyethylene glycosylated creatine, or creatine monohydrate, is believed to improve anaerobic performance and body composition. Theoretically, creatine used in combination with magnesium may enhance the production of energy by providing higher levels of adenosine triphosphate or ATP. It is believed that magnesium-creatine chelate may help runners delay exhaustion.

It may also be desired for the protein source of the present disclosure to impart beneficial health effects other than improved muscle mass, metabolism, and antioxidative properties. For instance, in one embodiment, ingredients that have anti-carcinogenic, anti-cardio disease, and anti-diabetic properties may be used. In one embodiment, a phytoalexin may be employed. Phytoalexins are antimicrobial and often antioxidative substances synthesized de novo by plants that accumulate rapidly at areas of pathogen infection. They are broad spectrum inhibitors and are chemically diverse with different types characteristic of particular plant species. Phytoalexins tend to fall into several classes including terpenoids, glycosteroids and alkaloids.

In a further embodiment, Trans-3,4′,5-trihydroxystilbene, better known as “Trans-Resveratrol”, a phytoalexin with chemopreventive, anticarcinogenic, cardioprotective, and anti-diabetic effects may be used. Trans-Resveratrol may be found in the skin of grapes and red wine. The chemical structure of Trans-Resveratrol is shown below:

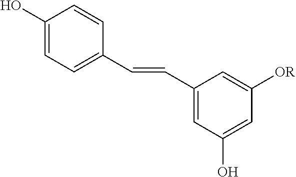

Trans-Resveratrol has anti-carcinogenic and chemoprotective properties. Trans-Resveratrol modulates signaling pathways that control cellular division, growth, apoptosis, inflammation, angiogenesis, and metastasis. Thus, Trans-Resveratrol has the distinct ability to affect all three stages of carcinogenesis: initiation, promotion, and progression. These effects reduce skin tumor formation and growth, inhibit mammary tumors, suppress prostate cancer progression, and liver cancer in rat and mice models. Recently, a 10-year epidemiological study on the relationships between Trans-Resveratrol supplementation and breast cancer indicated a 50% reduction in breast cancer risk when women consumed Trans-Resveratrol from grapes.

Trans-Resveratrol has long been used in oriental folk medicine for many therapeutic indications, including heart disease. In fact, the “French paradox” (i.e., a low mortality rate due to coronary heart disease despite high fat diets and high smoking rates), has been attributed to the routine consumption of red wine, and thus the high consumption of Trans-Resveratrol. Despite a lack of in vitro research in humans, animal models have been used to suggest that Trans-Resveratrol has cardioprotective effects by inhibiting low density lipoprotein oxidation, see Graph 1, inhibiting platelet accumulation, enhanced endothelial function and increased vasorelaxation.

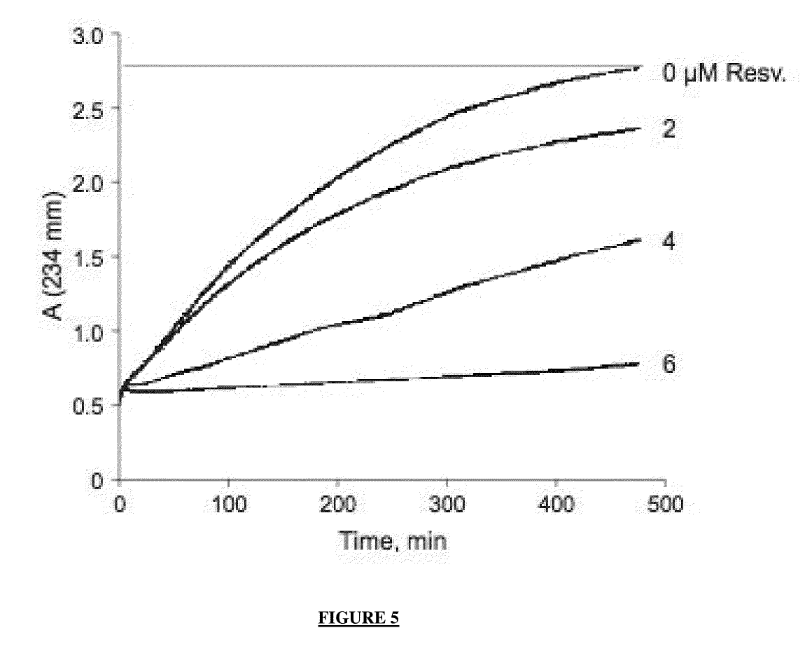

Graph 1, see FIG. 5, shows the effects of different concentrations of Trans-Resveratrol on low density lipoprotein oxidation. As Trans-Resveratrol concentrations increase, low density lipoprotein oxidation rates decreases.

Trans-Resveratrol is believed to have anti-hyperglycemic effects through stimulation of intracellular glucose transport. In fact, Trans-Resveratrol may promote the uptake of glucose in the absence of insulin. Diabetics who ingested Trans-Resveratrol had increased expression GLUT-4, an insulin-dependent glucose transporter. Not only does Trans-Resveratrol improve glucose uptake, it may also improve the action of insulin. Thus, a human body may dispose of/utilize glucose without massive releases of insulin into the blood stream.

The protein source of the present disclosure may also be enhanced by the addition of amino acids. For instance, the following amino acids may be employed, including but not limited to: alpha-ketoisocaporoic acid, L-Glutamine, L-Arginine, Beta-alanine, citrulline, L-Isoleucine, L-Leucine, L-Lysine, L-Methionine, L-Phenylalanine, L-Threonine, L-Tryptophan, L-Valine, L-Carnitine, choline, Acetyl-L-Carnitine, S-adenosyl-L-methionine, S-adenosyl-I-Methionine, acetyl-L-carnitine, L-citrulline, citrulline malate, L-Cysteine, N-acetylcysteine, alpha-ketoglutarate, histidine, L-Carnosine, beta-alanyl-I-histidine, beta-alanylhistamine, tyrosine, proline, hydroxyproline, alanine, asparagine, aspartic acid, D-aspartic acid, citrulline malate, glutamic acid, ornithine, ornithine alpha-ketoglutarate, serine, phosphatidylserine, glycine, taurine, and L-DOPA/Levodopa/3,4-dihydroxy-1-phenylalanine/clonidine.

In a further embodiment, Alpha-Ketoisocaproate (KIC), a product of L-leucine metabolism is further metabolized to HMB and may be used to enhance the current disclosure. Alpha-Ketoisocaproate may promote anabolic states by acting as an anti-catabolic agent. KIC may decrease muscle degradation following exercise without significantly stimulating anabolic signal transduction pathways. Supplements containing KIC in pharmacological doses are believed to decrease the rate of 3-methylhistidine excretions by patients with Duchene's muscular dystrophy. 3-methylhistidine is commonly used as an indicator of muscle protein breakdown or muscle wasting. It is believed that KIC promotes anabolic drive via stimulation of insulin secretion without table sugar. Other amino acid compounds have been shown to have the same effect. For example, arginine, ornithine, and ornithine alpha-ketoglutarate all stimulate insulin secretion. Insulin increases the intercellular transport of amino acids, and has both anabolic and anticatabolic effects.

In one embodiment, L-Alanyl-L-Glutamine may be used. L-Alanyl-L-Glutamine is the most abundant amino acid in the body and is converted to glucose when energy expenditure increases. In addition, it may be supplemented to clear ammonia in the bloodstream. Ammonia is toxic to animal tissues and increases during exercise. L-Glutamine provides fuel for certain cells of the immune system and is often used for nutritional and immune support by the physically active. It provides fuel for certain cells of the immune system and is often used for nutritional and immune support by the physically active. Endurance athletes undergoing rigorous, prolonged exercise may have an increased risk of infection due to immunosuppression and several clinical trials have reported that supplementation with L-Alanyl-L-Glutamine may decrease the incidence of illness in endurance athletes.

In a further embodiment, L-Arginine may be employed. L-Arginine, another amino acid, is believed to increase endurance and facilitate increases in anaerobic work capacity, may reduce blood toxins, and may increase muscle mass in resistance training athletes. For example, it is believed that in maximal exercise situations, L-Arginine may reduce peak plasma levels of the toxins ammonia and lactate.

In a further embodiment, the protein source of the present disclosure may include magnesium. Magnesium mobilizes the body's B vitamins and is a part of proteins, fatty acids and ATP synthesis. Magnesium is believed to regulate membrane stability and neuromuscular, cardiovascular, immune, and hormonal functions and is also believed to be a critical cofactor in numerous metabolic reactions. Deprivation of the mineral through exercise or physical activity along with marginal dietary intake may impair energy metabolism efficiency and the capacity for efficient physical work

In a further embodiment, the dietary nutritional supplement beta-hydroxy-beta-methylbutyrate (“HMB”) may be used. HMB is a commercially available dietary nutritional supplement believed to build muscle and strength plus promote fat reduction. HMB has become one of the most popular sports supplements on the market. A recent survey of 263 male college football players in the U.S. found 16% of athletes reported previous use of HMB, the sixth most commonly used supplement in this athletic group (Slater and Jenkins, 2000). In addition, HMB is believed to have anticatabolic effects of L-Leucine and its metabolites. The supplement is also believed to increase strength and lean body mass by acting as an anticatabolic agent, decreasing protein breakdown and damage to cells which typically occurs with intense exercise.

In a further embodiment, the protein source of the present disclosure may contain an enriched protein. This includes, but is not limited to: (1) albumin; (2) bovine colostrum; (3) whey protein isolate; (4) whey protein concentrate; (5) casein protein isolate/concentrate; (6) soy protein isolate/concentrate; (7) hemp protein isolate/concentrate; (8) vegetable protein isolate/concentrate; (9) egg protein isolate/concentrate; (10) beef protein isolate/concentrate; and (11) quinoa isolate/concentrate.

In a further embodiment, the enriched protein is L-Leucine, which has been shown to increase lean muscle mass more rapidly than other amino acids and to decrease muscle wasting and muscle soreness after strenuous activities. It may also improve immune system function and increase stamina faster than supplementing with only whole proteins. Further, L-Leucine enriched proteins allow these positive effects to occur in the absence of sugar. Use of L-Leucine may also reduce the amount of other proteins needed in the composition. For purposes of example only and not intended to be limiting, an optimal dose of Whey Protein for promoting muscle protein synthesis (“MPS”) is about 20 to 25 grams. However, a suboptimal dose of 6.25 g of Whey Protein, when supplemented with L-Leucine, stimulates MPS similar to a 25 g dose stimulating muscle growth while reducing caloric intake.

In a further embodiment the protein source contains an essential amino acid enriched with L-Leucine. This may make the product more effective and faster acting. In a further embodiment, the protein source contains an essential amino acid enriched with L-Leucine, as well as whole proteins, vitamins and minerals. These ingredients in combination may allow the protein source to not only be extremely effective, but it may also be fast acting on skeletal muscle. Furthermore, with regular exercise, the protein source of this embodiment may help reduce percent body fat and improve the user's overall health.

Other possible enrichments for the current disclosure include, but are not limited to, citrus aurantium, guarana, bitter orange, green tea, ginseng, taurine, caffeine, caffeine anhydrous, curicumin, black pepper, polyphenol complex, forskolin, rhodiola rosea, pantothenic acid (coenzyme A), biotin, vitamins A (retinoids), beta-carotene, 7-dehydrocholesterol, vitamin D2 (ergocalciferol), vitamin E (tocopherols and tocotrienols; alpha-tocopherol, beta-tocopherol, gamma-tocopherol, and delta-tocopherols), vitamin B1 (thiamine), vitamin B2 (riboflavin), fluoride, arsenic, boron, nickel, silicon, vanadium, vitamin B3 (niacin/nicotinic acid), niacinamide, vitamin B6 (pyridoxine, pyridoxal and pyridoxamine), vitamin B12 (cobalamin), vitamin C (ascorbic acid), vitamin D, vitamin D3 (as cholecalciferol), (wheat germ, safflower oils, safflower oils enriched with alpha-tocopherol, vitamin K (phylloquinones and menaquinones; menadione, phylloquinone, menaquinone-7), phosphorus, sulfur, folate, magnesium, chloride, calcium carbonate, iron, zinc, chromium, nitric oxide, carotenoids (lutein and zeaxanthin), lycopene, coca extract, probiotics (lactobacillus acidophilus, L. bulgaricus, L. reuteri, L. casei, L. lactis, B. lactis, L. rhamnosus, B. bacterium longum, Leuconostoc cremoris, B. bacterium breve, S. diacetylactis, S. diacetylactis, S. florentinus, L. plantarum), soluble/insoluble fibers (prebotics), inulin, angelica sinensis (angelica root, Angelique, dong quai, engelwurzel, garden angelica, heiligenwurzel, root of the Holy Ghost, Tang-Kuel, Wild Angelica), melatonin, valerian root, glucosamine, chondroitin sulfate, horneygoatweed sodium, potassium, sodium bi-carbonate, chromium, copper, iodine, Iron, manganese, molybdenum, selenium, zinc, vanadium, nickel, boron, silicon, arsenic, fluorine, xanthohumol (XN (2.5-20 micromole), trans-resveratrol, French maritime bark extract, omege-3/fish oil (eicosapentaenoic acid 20:5 delta 5,8,11,14, 17 n-3 from fish, dairy products, and shellfish), docosahexaenoic acid 22:6 delta 4,7,10,13,16,19 (n-3) (from fish, dairy products, and shellfish), linoleic acid 18:2 delta 9,12 n-6 delta 9,12,15 (from corn, safflower, soybean, cottonseed, sunflower seed, peanut oils, and acai berry), alpha-linolenic acid 18:3 delta 9,12,15 n-3 (from soy, canola, linseed, rapeseed, other seed oils, and leafy vegetables), omega-6/conjugated linoleic fatty acids, arachidonic acid 20:4 delta 5,8,11,14 (from animal fats), phosphatidylserine, albumin, bovine colostrum, choline, whey protein isolate, whey protein concentrate, casein protein isolate/concentrate, soy protein isolate/concentrate, hemp protein isolate/concentrate, vegetable protein isolate/concentrate, egg protein isolate/concentrate, beef protein isolate/concentrate, high fructose corn syrup, cane sugar, beet sugar, maltodextrin, microcrystalline cellulose, corn maltodextrin, creatine monohydrate, creatine HCL, alpha-ketoisocaproic acid, L-glutamine, L-Arginine, Beta-alanine, citrulline, superoxide dismutase (SOD), L-isoleucine, L-Leucine, L-lysine, L-methionine, L-phenylalanine, L-Threonine, L-Tryptophan, L-Valine, L-Carnitine, choline, Acetyl-L-Carnitine, S-adenosyl-L-methionine, S-adenosyl-I-methionine, acetyl-L-carnitine, L-citrulline, citrulline malate, L-cysteine, N-acetylcysteine, histidine, L-carnosine, beta-alanyl-I-histidine, beta-alanylhistamine, tyrosine, proline, hydroxyproline, alanine, asparagine, aspartic acid, D-aspartic acid, glutamic acid, ornithine, ornithine alpha-ketoglutarate, serine, phosphatidylserine, glycine, taurine, L-DOPA/Levodopa/3,4-dihydroxy-1-phenylalanine/clonidine, citrus aurantium, guarana, bitter orange, green tea, cardamom, carline thistle, castor bean, catnip, Cat's-Claw, clandine, celery, centaury, chamomile, chaparral (Larrea tridentate, L. divaricata), chaste tree (Vitex agnus-castus), chaulmoogra oil (Hydnocarpus wight-iana, H. anthelmintica, and Taraktogenos kurzii), chickweed (Stellaria media), chicory (Cichorium intybus), Chinese rhubarb (Rheum palmatum), cinnamon (C. zeylandicum, C. loureirii, C. zeylanium, and various species of Cinnamomum), clary (Salvia sclarea), cloves (Eugenia caryophyllata, Caryophyllus aromaticus), coenzyme Q10 (2,3 dimethoxy-5 methyl-6-decaprenyl benzoquinone), coffee, cola tree (Cola nitida, C. acuminata), coltsfoot (Tussilago farfara), comfrey (Symphytum officinale), condurango (Marsedenia condurango), coriander (Coriandrum sativum, C. sativum var. vulgare, C. sativum var. microcarpum), corkwood (Duboisia myoporoides), couchgrass (Agropyron repens), cranberry (Vaccinium macrocarpon, V. oxycoccus, V. erythrocarpum), cucumber (Cucumis sativus), daffodil (Narcissus pseudonarcissus), daisy (Bellis perennis), damiana (Turnera diffusa), dandelion (Taraxacum officinale, T. laevigatum), devil's claw (Harpagophytum procumbens), dehydroepiandrosterone (DHEA), dill (Anethum graveolens), dock yellow (Rumex crispus), dong quai (Angelica polymorpha var. sinensis), echinacea (Echinacea angustifolia, E. pallida, E. purpurea), elderberry (Sambucus, S. nigra, S. Canadensis), elecampane (Inula helenium), brigham tea, cao ma huang, desert tea, epitonin, herbal, joint fir, ma huang, mahuuanggen root, Mexican tea, mormon tea, muzei mu huang, natural ecstacy, popotillo, sea grape, squaw tea zhong ma huang, eucalyptus, eyebright (Euphrasia officinalis), false unicorn root (Chamaelirium luteum), fennel (Foeniculum vulgare), fenugreek (Trigonella foenum-graecum), feverfew (Chrysanthemum parthenium, Tanacetum parthenium, Matricaria parthenium, Leucanthemum parthenium, Pyrethrum parthenium), figwort (Scrophularia nodosa, S. ningpoensis), fumitory (Fumaria officinalis), galangal (Alpinia officinarum), galanthamine (Galanthus nivalis), garlic (Allium sativum), gentian (Gentiana lutea), ginger (Zingiber officinale), ginkgo (Gingko biloba, Salisburia adiantifolia), ginseng (Panax quinquefolius), ginseng Siberian (Eleutherococcus senticosus), glucomannan (Amorphophallus konjac), goat's rue (Galega officinalis), goldenrod (Solidago virgaurea), goldenseal (Hydrastis Canadensis), gossypol (Gossypium hirsutum), gotu kola (Centella asiatica), grape seed/pinebark (Vitis vinifera, Pinus maritime, Pinus nigra), green tea (Camellia sinensis), gum arabic (Acacia Senegal), hawthorn (C. laevigata, C. monogyna, C. folium), Hellebore American (Veratrum viride, V. album, V. californicum, V. officinale, V. japonicum), hops (Humulus lupulus, Cannabis sativa/indica), horehound (Marrubium vulgare), horse chestnut (Aesculus hippocastanum), horseradish (Armoracia rusticana), horsetail (Equisetum arvense, Equisetum), hyssop (Hyssopus officinalis), indigo (Indigofera, I. tinctoria, I. suffruticosa), Jamaican dogwood (Piscidia erythrina), jambul (Syzygium cuminii), jojoba (Simmondsia chinesis, S. californica), juniper (Juniperus communis), karaya gum (Sterculia urens), kava (Piper methysticum), kelp (Laminaria digitata, L. japonica, L. saccharina, Macrocystis pyrifera), kelpware (Fucus vesiculosus), khat (Catha edulis), khella (Ammi visnaga), lady's slipper/yellow (Cypripedium pubescens, C. calceolus, C. parviflorum), lavender (Lavandula officinalis, L. latifolia, L. angustifolia, L. stoechas), licorice (Glycyrrhiza glabra), lily of the valley (Convallaria majalis), lobelia (Lobelia inflata, L. berlandieri, L. cardinalis, L. inflata, L. siphilitica), lovage (Levisticum officinale, L. radix), madder (Rubia tinctorum), male fern (Dryopterir filix-mas), mallow (Malva sylvestris), marjoram (Origanum majorana, O. vulgare, Thymus mastichina), marshmallow (Althaea officinalis), mayapple (Podophyllum peltatum, Mandragora officinarum), meadowsweet (Filipendula ulmaria), melatonin (n-acetyl-5-methoxytryptamine), milk thistle (Silybum marianum), mint (Mentha×piperita, Mentha spicata, M. aquatica), mistletoe (Viscum album, V. abietis, V. austriacum, Phoradendron flavescens, P. serotinum, P. tomentosum), motherwort (Leonurus cardiaca, L. Artemisia, L. glaucescens, L. heterophyllus, L. quinquelobatus, L. sibiricus), mullein (Verbascum Thapsus), myrrh (Commiphora molmol, Commiphora), myrtle (Myrtus communis), nettle (Urtica dioica), night-blooming cereus (Selenicereus grandiflorus), nutmeg (Myristica fragrans), oaks (Quercus robur, Q. petraea, Cynips), oats (Avena sativa), octacosanol (Eupolyphaga sinensis, Acacia modesta, Serenoa repens), oleander (Nerium oleander), oregano (Origanum vulgar, O. majorana), oregon grape (Mahonia aquifolium, Berberis vulgaris), pansy (Viola tricolor), papaya (Carica papaya), pareira (Chondrodendron tomentosum), parsley (Petroselinum crispum, Apium petroselinum, Carum petroselinum, Petroselinum sativum), parsley piert (Aphanes arvensis, Alchemilla arvensis, A. microcarpa), passion flower (Passiflora incarnata, P. caerulea), pau D'arco (Tabebuia impetiginosa, T. avellanedae, Tecoma curialis), peach (Prunus persica, Prunus armeniaca), pennyroyal (hedeoma pulegioides, Mentha pulegium), peyote (Lophophora williamsii), pill-bearing spurge (Euphorbia pilulifera, E. hirta, E. capitata), pineapple (Ananas comosus), pipsissewa (Chimaphila umbellate), plantains (Plantago lanceolata, P. major, P. psyllium, P. ovata), pokeweed (Phytolacca Americana), pomegranate (Punica granatum), poplar (Populus alba, P. tremuloides, P. nigra), prickly ash (Zanthoxylum americanum, Z. clava-herculis), primrose/evening (Oenothera biennis), pulsatilla (Anemone pulsatilla, Pulsatilla vulgaris), pumpkin (Cucurbita pepo, C. maxima, C. moschata, C. pepo), Queen anne's lace (Daucus carota), Quince (Cydonia oblonga), raspberry (Rubus idaeus), rauwolfia (Rauvolfia serpentina), red clover (trifolium pratense), red poppy (Papaver rhoeas), rhatany (Krameria triandra), rose hips (Rosa canina), rosemary (Rosemarinus officinalis), royal jelly (Apis mellifers), rue (Ruta graveolens), safflower (Carthamus tinctorius), Saffron (Crocus sativus), sage (Salvia officinalis), St. John's Wort (Hypericum perforatum), santonica (Artemesia cina), Sarsaparilla (Smilax aristochiifolia, S. regelii, S. febrifuga, S. ornate), sassafras (Sassafras albidum), saw palmetto (Serenoa repens, Sabal serrulata), schisandra (Schisandra chinesis), sea holly (Eryngium maritimum), self-heal (Prunella vulgaris), senega (Polygala senega), senna (Cassia acutifolia, C. augustifolia, C. senna, C. obovata, C. lanceolata, C. marilandica, C. chamecrista, C. fistula), shark cartilage (Squalus acanthias, Sphyrna lewini), shepherd's purse (Capsella bursa-pastoris), skullcap (Scutellaria laterifolia, S. baicalensis), skunk cabbage (Symplocarpus foetidus), slippery elm (Ulmus rubra Muhl, Ulmus fulva Mich.), soapwort (Saponaria officinalis), southernwood, spirulina (all 35 Spirulina species that appear in chlorophyll and phycocyanin pigments), squaw vine (Mitchella repens), squill (Urginea maritime), stoneroot (Collinsonia Canadensis), sundew (Drosera rotundifolia), sweet cicely (Myrrhis odorata), sweet violet (Viola odorata), tansy (Tanacetum vulgare, Senecio jacobaea), tea tree (Melaleuca alternifolia), thuja (Thuja occidentqlis), thyme (Thymus vulgaris), tonka bean (Dipteryx odorata), tormentil (Potentilla tormentilla), tragacanth (Astragalus gummifer), true unicorn root (Aletris farinose), Turmeric (Curcuma longa), valerian (Valeriana officinalis), vervain (Verbena officinalis), wahoo (Euonymus atropupureus), watercress (Nasturtium officinale), wild cherry (Prunus virginiana, P. serotina), wild ginger (Asarum canadense), wild indigo (Baptisia tinctoria), wild lettuce (Lactuca virosa), wild yam (Dioscorea villosa), willow (Salix alba, S. nigra), wintergreen (Gaultheria procumbens), witch hazel (Hamamelis virginiana), wormwood (Artemisia absinthium), woundwort (Stachys palustris, S. sylvatica), yerba mate (Ilex paraguariensis), yerba santa (Eriodictyon californicum), yew (Taxus brevifolia), and yohimbe (Pausinystalia yohimbe, rauwolfia serpentina), as well as targeting drugs to inhibit glycation (protein), including but not limited to: chelators—diethylene triamine pentaacetic acid, 2) reducing agents—glutathione, 3) radical scavengers—aspirin, flax, anthocyanins, hydroxycinnamates, flavan 3-ols, flavonols (chlorogenic acid, neochlorogenic acid, catechin, epicatechin, and quetcetin 3-rutinoside), cyaniding-3-glucoside, and cyanidin.

In a yet other embodiment, quercetin may be employed in the combination. Quercetin may be found in food plants, such as, berries, apples, cranberries, onions, citrus and various other fruits, leafy vegetables, roots, tubers and bulbs, herbs and spices, legumes, and cereal grains, as well as in tea, black tea, and cocoa. Quercetin is believed to have anti-oxidant, anti-carcinogenic, anti-inflammatory, cardioprotective, anti-inflammatory, and free radical scavenging properties of quercetin and is not believed to have adverse health effects when ingested.

In a further embodiment, antioxidants may be employed. In a still further embodiment, Vitamins C and E, alone or in combination, may be employed as antioxidants that may prevent exercise induced oxidative stress. Vitamin C and vitamin E are believed to decrease the exercise-induced increases in fatty acid metabolism. Vitamin C (ascorbic acid) is involved in a number of biochemical pathways that enhance exercise and overall health. Vitamin C is believed to inhibit lipid oxidation in human HDL (high density lipoprotein cholesterol remnant) or good cholesterol, thereby, preserving the cardioprotective antioxidant function of HDL. Furthermore, levels of erythrocyte (red blood cell), antioxidant enzymes, and plasma antioxidants during exercise improved following Vitamin C supplementation.

Vitamin E, including but not limited to tocopherols and tocotrienols; alpha-tocopherol, beta-tocopherol, gamma-tocopherol, and delta-tocopherols, may protect against free radicals, which are produced from exercise. Vitamin E is believed to significantly increase circulating neutrophils in older, but not younger, subjects performing eccentric exercise that causes an increase in skeletal muscle damage. In addition to its augmenting effects on neutrophil migration to sites of exercise induced muscle damage, vitamin E may cause a greater increase in circulating creatine kinase (CK) activity, another marker of cellular damage, thus indicating skeletal muscle repair. In addition, vitamin E consumption is believed to be associated with enhanced glucose tolerance and insulin sensitivity as well as improved lipoprotein status. Vitamin E may also enhance endurance and help to prevent the production of oxidized low-density lipoprotein (LDL) cholesterol caused by strenuous endurance exercise.

In a still further preferred embodiment, the protein source may be formulated to increase muscle mass, improve body composition, increase insulin sensitivity, fight inflammation and fatigue, fight cancer, and protect the user's cardiovascular system. In a still further preferred embodiment, the protein source comprises whole proteins, BCAAs, essential amino acids, essential amino acid metabolites, vitamins and minerals, and Trans-Resveratrol.

Various forms of delivery are possible for the protein source. Oral and intravenous administration are possible with targets of action that are topical (local), enteral (system-wide effect, but delivered through the gastrointestinal tract), or parenteral (systemic action, but delivered by routes other than the GI tract). Indeed, possible ways of administering the protein source include oral, topical, inhalational, buccal, sublingual, nasal, suppository, parenteral or injection. In a preferred embodiment, the protein source is delivered orally. When delivered orally, dosage forms of the protein source may include liquid, solid, and semisolid dosage forms. In a preferred embodiment, the dosage form is a solid or semisolid.

Common solid dosage forms include pills, tablets, and capsules. In a further preferred embodiment, the dosage form is a semi-solid such as a “gummy” product. In a still further embodiment, the gummy may be a gelatin-based, chewy candy. The gummy dosage may include a mixture of sugar, glucose syrup, starch, flavoring, food coloring, citric acid, and gelatin. Sugar free and organic candy, suitable for vegetarians, or those following religious dietary laws are also within the scope of this disclosure.

In a further example a gummy matrix may be formed. In one such embodiment, the gummy matrix is a mixed gel. In a further embodiment, the mixed gel may comprise gelatin at 1 to 10 parts, gellan gum 0.099 to 1.5 parts, pectin 0.19 to 1.5 parts, modified starch at 1 to 5 parts, and fructooligosaccharides 1 to 5 parts.

In a further embodiment, sugar may be added to the gummy matrix. When sugar is added the gummy matrix may consists of gelatin 3 to 11 parts, gellan gum 0.09 to 2 parts, pectin 0.19 to 1.3 parts, modified starch 1 to 5 parts, and fructooligosaccharides 1 to 5 parts. Artificial sweeteners may be used instead, or in addition to, sugar. When artificial sweeteners are added gelatin consists of 2 to 10 parts, gellan gum 1 to 1.5 parts, pectin 1 to 1.5 parts, modified starch at 1 to 5 parts, and fructooligosacahrides 1 to 5 parts.

During processing, the mixing temperature should be raised to, at least, a range between 59° C. to 79.9° C., and then gelatin should be added. The gelatin should be stirred until it is completely dissolved, standing at the same temperature for a range of 1 to 3.5 hours. Other possible ingredients (e.g., gellan gum and pectin with cane or beet sugars or the listed artificial sweeteners (e.g., sugar alcohols) may be added in an amount 0.1 to 5 times the weight of the solution. The mixture is slow boiled until the final solution is ready. Slow boiling may include, raising the temperature of the mixture to 60 C, adding pre-mixed ingredients, such as whey, casein, leucine, gelatin, Stevie, sucralose, sorbital, oil, beeswax, carnuba wax, and Vitamin C. The mixture may be mixed vigorously until completely dissolved but mixing should be used for five to fifteen minutes, with fifteen minutes being the maximum mixing time. Oil and beeswax may be added with the mixture remaining at 60 C and again mixed vigorously. Citric Acid and Vitamin C may be added and also mixed vigorously, at this time the temperature should be reduced to below 45 C. At this point, the mixture may be put into molds and completely cooled to 0.6 C.

Once the gummy matrix is ready, sugars can be added following a melting phase in water-dispersed, modified starch paste in an amount at least twice the weight thereof. The sugar solution should boil until the solution reaches 70 to 81 Brix as measured by Brix spindle and a vacuum degassing should be performed after complete mixing of ingredients so the solution contains no bubbles. The mixed solution should be 79.9° C. to 96° C. and cooled to 40-45° C. The active ergogenic ingredients may be mixed and blended at this point and poured into a blending barrel. Other ingredients, such as flavor agents, such as sour agents, edible essence, edible colorant, etc., may be added to the mixed solution. The solution should then be homogenously stirred and blended and the mixed gummy solution should next be casted/molded by transferring the blended solution into a casting machine.

Additional ingredients, including but not limited to the following may also be added to the gummy matrix: beta-glucans, dextrans, dextrins/maltodextrins, guar, locust bean, plant extracts (Arabic and tragacanth), carrageenan (seaweed extract, eucheuma cottonii, eucheuma spinosum, gigartina species), Irish Moss, Eucheuma species), brand sta-slim (supplier A. E. Staley, modified potato starch), brand stellar (supplier A. E. Staley, acid modified corn starch), brand Amalean 1 (supplier Am. Maize, modified high amylose corn starch), brand Paselli SA2 (supplier Avebe America Inc., potato maltodextrin), brand oatrim (supplier conagra, Quaker Oats, oat maltodextrin), brand novagel (supplier FMC, microcrystalline cellulose), brand maltrin (supplier grane processing, corn maltodextrin), brand splendid (supplier Hercules, Inc., citrus pectin), brand n-oil (supplier Nat. Starch, tapioca dextrin), brand litesse (supplier Pfizer, polydextrose), brand ricetrinthree (3) (supplier Zumbro, rice maltodextrin).

In a further embodiment, the protein source is a gummy product that may employ micro-encapsulation technology for extended release of important synergistic ingredients infused within a gummy dosage form. In one embodiment, beta-hydroxy-beta-methylbutyrate (“HMB”) microspheres may be employed.

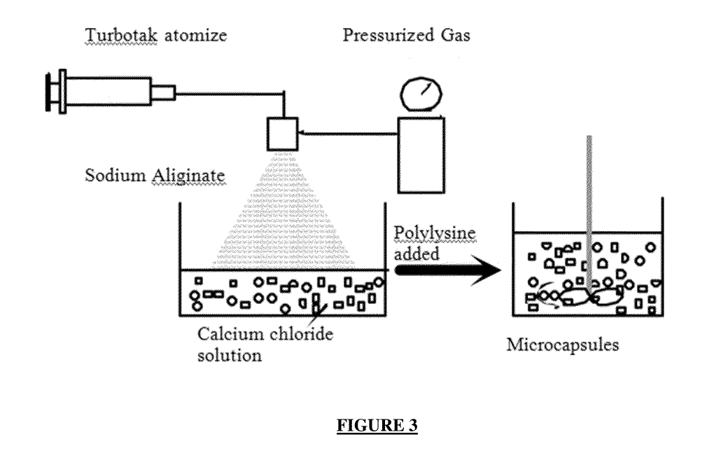

Microencapsulation is the process of enclosing these ingredients into a cell by coating them with a proper substance in a way that results in appropriate cell release in the intestinal medium. For purposes of example only and not intended to be limiting, HMB and KIC microspheres may be prepared through an ionic gelation process using sodium alginate as a polymer. For purposes of example only and not intended to be limiting, HMB microspheres were prepared through an ionic gelation process using sodium alginate as a polymer. See FIG. 3. The sodium alginate polymer was dispersed in a required amount of distilled water to form a homogenous polymer mixture. The amount of water in the gummy matrix may be determined by adding twice the amount of water to the weight of the prepared gelatin. In a further embodiment, water may be added in a range from 5 to 12 times the weight of the prepared gelatin. In a further embodiment, water should be at least 10% to 20% of the gelatin weight. In a more preferred range, water may be 14% to 18% of total prepared gelatin weight. HMB was added to the polymer dispersion and mixed thoroughly with a magnetic stirrer to form a viscous homogenous dispersion. The resulting dispersion was then added manually drop wise into calcium chloride (10% w/v) solution (40 ml) through a syringe with a needle of size no. 20. The added droplets were retained in the calcium chloride solution for 30 minutes to complete the curing reaction and to produce spherical rigid microcapsules having a different coat: core ratio (1:1).

It is possible that time-release KIC and HMB microcapsules may have incomplete forms. By increasing the polyelectrolyte complex with oppositely charged polyelectrolytes, however, the mechanical strength of the hydrogel and/or permeability barrier may be enhanced. For example, addition of a polycation (such as but not limited to poly-L-lysine) allows a membrane of polyelectrolyte complex to form on the surface of alginate beads.

In further preferred embodiment, the protein source may include artificial sweeteners such as D-sorbitol, D-mannitol, erythritol, xylitol, thaumatins, glycyrrhizin, stevioside, tagatose, acesulfame potassium, alitame, saccharin, dextrose, neotame, acesulfame K (Aka Sunette, Sweet One, Sweet n Safe Acesulfame-k), citrus pectin, tapioca dextrin, polydextrose, rice maltodextrin, nutrasweet (brand simplesse), nutrasweet (brand simplesse 100), national starch (brand n-flate), Nabisco (brand salatrim), proctor & Gamble (brand caprenin), Proctor & Gamble (brand Olestra), aspartame, stevie, sucralose, maltodextrose, sucrose, high fructose corn syrup. In one embodiment, the amount of sweetener may range from 1 to 50 grams per batch and may be scaled up to 50,000 kilograms for manufacturers. In a preferred embodiment, the amount of sweetener may range from 1 to 10 grams per batch.

The protein source may also include whole proteins. Suitable whole proteins include but are not limited to whey protein isolate, soy protein, casein protein, egg protein, beef protein, and hemp protein. The amount of whole protein may range from 1 to 10 grams. The protein source may also include Branched Chain Amino Acids. These include but are not limited to L-Leucine, L-Isoleucine, and L-Valine. In one embodiment, the branched chain amino acids may be present from 1 to 50 grams per batch and scaled up to 50,000 kilograms for manufacturers. In a preferred embodiment, the branched chain amino acids may be present from 1 to 8 grams. The protein source may also include L-alanyl-L-glutamine that may be present from 1 to 50 grams per serving and scaled up to 50,000 kilograms for manufacturers. In a preferred embodiment, L-alanyl-L-glutamine may be present from 1 to 8 grams per serving. This embodiment may also include essential amino acids including but not limited to L-Histidine, L-Lysine, L-Methionine, L-Phenylalanine, and L-Threonine. These may be present from 1 to 50 grams per serving and scaled up to 50,000 kilograms for manufactures. In a preferred embodiment, they are present from 1 to 10 grams. This embodiment may also include L-Arginine from 1 to 50 grams per serving and scaled up to 50,000 kilograms for manufactures. In a preferred embodiment, L-Arginine is present from 1 to 10 grams. Trans-Resveratrol from 1 to 50 grams per serving and scaled up to 50,000 kilograms for manufactures. In a preferred embodiment, Trans-Resveratrol is present from 1 to 10 grams. Microencapsulated HMB may be present from 1 to 50 grams per serving and scaled up to 50,000 kilograms for manufactures. In a preferred embodiment, Microencapsulated HMB is present from 1 to 10 grams. This embodiment may also include a Gummy Solution. The Gummy solution may include but is not limited to gelatin, potassium, sodium, chloride, magnesium, citric acid present at 85% by weight.

A further embodiment provides mixing branched chain amino acids and trans-resveratrol for at least 15 to 45 minutes, preferably from 15 to 30 minutes, while cooling temperatures are below 150° C. and closer to 96° C. This method includes mixing whole proteins for less than 15 minutes after gummy matrix temperature is reduced from 60.25° C. and closer to 40-45° C. The resulting gummy matrix may be stored at 15° C. to 29.9° C. at a relative humidity of 10% to 41%, and drying the product to a water content of 8% to 21%. As used herein, “%” refers to “% by mass”, unless otherwise specified. This embodiment may also include adding microencapsulated HMB during the cooling phase. Cinnamon extract flavoring may be used to enhance flavor.

Also included in this disclosure is a method of making an improved protein source. The method includes preparing whole proteins at a temperature range of 90 to 113° F. for less than 15:00 minutes. This embodiment may also include preparing branched chain amino Acids, L-alanyl-L-glutamine, and essential amino acids, L-Arginine, and Trans-Resveratrol: 100 to 284 degrees Fahrenheit for less than 30 minutes. This embodiment may also include adding microencapsulated HMB during the cooling phase. In a further embodiment, the branched chain amino acids, L-alanyl-L-glutamine, essential amino acids, L-Arginine, Trans-Resveratrol may be combined in the stirring chamber prior to adding the whole proteins. Microencapsulated HMB may then be added during the cooling phase.

The current disclosure promotes MPS, attenuates muscle protein degradation, and increases insulin blood levels in healthy individuals, improves muscular endurance at a high rate. The current disclosure may function synergistically with trans-resveratrol for decreasing the levels of cellular oxidative stress or cell damage from free radical formation. The current disclosure may also promote anti-aging as well as may protect from cancer cell proliferation. Moreover, the current disclosure provides a convenient delivery system that may be in the form of a gummy candy, as opposed to the drinks and powders used by others. The current disclosure may also be suitable for individuals with type 2 diabetes, or who are overweight and obese. Typically, these individuals are recommended to low glycemic diets with caloric restriction. In part, these designs, limit the consumption of foods with high amounts of table sugar or sucrose.

The present invention provides a ready-to-use enteral nutritional formulation having biological effects on humans or other animals. The system may provide complete nutrient requirements of athletes, health conscious individuals, and non-intensive cares patients (i.e., suffering from a variety of diseases or insults) who may have compromised absorption capacity and dietary reference intakes. The present invention supplements the unique nutrient needs of these individuals that are generated due to exercise, tissue repair, and healing requirements. The present invention may play a vital role in all biological processes to be used in treatment, cure, prevention, or diagnosis of disease or used to otherwise enhance physical or mental well-being. The system may also combat potential health threats.

To this end, the above objectives are accomplished according to the present invention by providing steps to process and manufacture a protein delivery system. The system may be a semi-solid. The composition preferably includes at least one protein source, at least one amino acid source, at least one amino acid metabolite source, at least one enzyme source, at least one lipid source, at least one vitamin, at least one mineral, at least one botanical/herbal-extract, at least one caloric intake reducing agent, and at least one antioxidant/anti-carcinogenic factor. Furthermore, the protein and amino acid sources may comprise of different combinations that are concentrated, isolated, and/or hydrolysated in composition; as well as, other free amino acids and their metabolites.

In one embodiment, a method for providing nutritional support is provided that provides a composition containing approximately 1% to 50% of a protein source(s), in other embodiments, the protein source(s) may be present from 25% to 30%, 16% to 22%, and 14% to 20%. In a further embodiment, an amino acid source(s) may be present from 1% to 15%, in a further embodiment, the amino acid source(s) is present from 10% to 15%, and in a further embodiment the amino acid source(s) is present from 3% to 9%. In another embodiment, a lipid source(s) comprising of omega 3 and omega 6 fatty acids may be present from 1% to 20%, from 10% to 15% in a further embodiment, and from 3% to 9% in yet another embodiment. In a further embodiment, an antioxidant/anti-carcinogenic source(s) may be present in various embodiments by 1% to 10%, from 0.9% to 0.3%, or from 0.2% to 0.8%. A multivitamin source may be present in various embodiments from 1% to 10%, from 0.9% to 3.0%, from 0.2% to 0.8% or at 1.2%. Combinations of the above ingredients at the various percentages are considered within the scope of the current disclosure. All percentages are by weight.

In respect to commercial processing and manufacturing techniques, if desired the present embodiments can incorporate other drugs and additive sources, and be produced using an array of commercial processing methods. These may include: sugar addition to increase gelation temperature and gel strength; adding polyol compounds or carbohydrate to improve heat-stability, decrease turbidity and/or gelation; adding sugar alcohols or amounts of sodium salts, aminopolycarboxylic acids, and protein hydrolysates sources to increase thermal denaturation temperatures; adding enzymatic cross-linking enzymes to increase heat stability and decrease gel strength; adding mineral chelators to reduce protein aggregation and gelation; adding molecular chaperones to increase heat stability, improve clarity, and prevent denaturation, aggregation, and precipitation/syneresis; enzymatic hydrolysis to modify foaming, gelling and emulsifying; ultrasonication to increase or decrease turbidity and improve heat stability and clarity; electrostatic repulsion to improve heat stability and clarity; prolease technology, nano-particulate and microparticulate technology, and mucoadhesive delivery for absorption capacity; protein encapsulation for delivery of micro/nano-particles as a delivery system of protein models, such as one-step and two-step desolvation techniques; formation of soluble aggregates to improve solubility and heat stability, decrease viscosity and turbidity, as well as, resulting in agglomeration and subsequent formation of particles.

Ultrasonication, a technique that uses acoustic cavitation, may be used to decrease turbidity in whey protein solution. In one embodiment, the method may be used at 15 W of electrical power, ranging from five to fifteen minutes, and at temperatures ranging from 20 C to 60 C.

In a preferred embodiment, the protein source may include at least 14% to 22% from protein hydrolysates blended with chelators and chaperones that may be processed using ultrasonication techniques, 5% to 7% from microemulsions, nanoparticles, and soluble aggregates, 13% to 16% from sugar and/or polyol compounds, and approximately 2% to 3% from lipid(s), 4% to 9% from fiber(s), 0.2% to 1.2% from sodium salts and aminopolycarboxylic acids, 2% to 8% enzymes, 9% to 30% from polysaccharides, by weight.

The aforementioned embodiments may include the following ingredients: Distilled water at 40.0% to 60.0%, Citric acid at 0.1% to 0.4%, Vitamin C, Whey protein isolate, Micellar casein protein, L-leucine, Gelatin collegen protein, Canola/olive oil, Beeswax at 0.1% to 2.0%, Stevie polyol compounds at 3.0% to 15.0%, Sorbital polyol compounds 3.0% to 15.0%, and/or Sucralose polyol compounds 3.0% to 15.0%.

In respect to commercial processing and manufacturing techniques, in one embodiment, sugar or polyol compounds may be added to increase gelation temperature and gel strength, as known to those of skill in the art. Moreover, carbohydrates may be added to improve heat-stability through conjugation of whey proteins. Further, Enzymatic cross-linking enzymes may be used to increase heat stability and decrease gel strength. Molecular chaperones, compounds that help stabilize whey proteins and prevent them from unfolding, aggregating and precipitating may also be used to produce the embodiments disclosed herein. Casein protein has been reported to act as a molecular chaperone by binding with other proteins to make them more heat-stable and more resistant to aggregation.

Furthermore, other free amino acids and amino sugars, and amino acid metabolites may be added to induce muscle protein synthesis, attenuate protein degradation and muscle weakness, and promote skin health. Preferably, the present embodiment(s) comprise at least 3% from an amino acid or amino acid metabolite source, by weight.

Other suitable amino acids may include: Conditionally Essential Amino Acids including: Arginine, Cysteine, cystine, Glutamine, Histidine, Proline, Taurine, Tyrosine; Nonessential Amino Acids including: Alanine, Asparagine, Aspartic acid, Citrulline, Glutamic acid, Glycine, Serine; and Essential amino acids, including: Isoleucine*, Leucine*, Lysine, Methionine, Phenylalanine, Threonine, Tryptophan, Valine*—*indicates branched chain amino acids.

Moreover, an advantage of the present invention is to provide a composition having high and effective protein content in conjunction with sufficient lipid, vitamin, mineral, botanical/herbal extract, caloric intake reducing agent, and antioxidant/anti-carcinogenic sources. Thus, the purpose of these embodiments is to supplement protein intake, while defending against deterioration of the composition and cellular damage from free radicals; enhance immune system and other physiological functions; decrease risk of certain cancers and cardiovascular disease; and promote healthy weight loss. Additionally, pursuant to the present invention, the use of certain components promotes healing, tissue repair, and decreases inflammatory reactions.

Yet another advantage of the present invention is to provide a composition that has reduced caloric density, water, and carbohydrate content, thus reducing the risk of hyperglycemia, and diarrhea due to carbohydrate intolerance, and over hydration.

Furthermore, another advantage of the present invention is that it utilizes compositions that may contain unique peptide profiles derived from pancreatic enzymes, and therefore, promote improved tolerance, palatability, absorption, nitrogen utilization and retention, and repletion of gut integrity. It has been shown that such pancreatic-derived peptides are more functional to human physiology than those produced with microbial enzymes.

Moreover, an advantage of the present invention is that the delivery system is semi-solid and not a powder. Thus, no mixing is required, and therefore, reduces the risk of bacterial contamination.

In another embodiment fiber may be included, such as Pectin (1 to 38 g), lignin (1 to 38 g), hydrocolloids (1 to 38 g), and fructans (i.e., inulin, oligofructose, and fructooligosaccharides) (1 to 38 g).

In another embodiment stimulants may be included such as citrus aurantium (1 to 300 mg), guarana (1 to 300 mg), bitter orange (1 to 300 mg), green tea (1 to 300 mg), ginseng (1 to 300 mg), taurine (1 to 300 mg), caffeine (1 to 300 mg), caffeine anhydrous (1 to 300 mg), curicumin (1 to 300 mg), black pepper (1 to 300 mg), polyphenol complex (1 to 300 mg), forskolin (1 to 300 mg), rhodiola rosea (1 to 300 mg), thiamin (0.2 to 1.4 mg), riboflavin (0.3 to 1.6 mg), niacin (2 to 35 mg), biotin (5 to 35 μgrams), vitamin B6 (1 to 100 mg), folate (1 to 1000 μgrams), and vitamin B12 (0.4 to 4.0 μgrams).

In another embodiment, thermogenics, as known to those of skill in the art may be incorporated into the protein sources.

In another embodiment, electrolytes such as sodium (120 to 1500 mg), potassium (400 to 2300 mg), and chloride (180 to 2300 mg) may be included.

In another embodiment, major minerals may be included such as Calcium (200 to 1300 mg), phosphorus (100 to 1250 mg), and magnesium (30 to 420 mg).

In another embodiment nonessential trace/ultratrace minerals may be included such as Fluoride (0.01 to 4 mg), Boron (3 to 20 mg), Nickel (0.2 to 1.0 mg), and Vanadium (0.01 to 1.8 mg).

In another embodiment essential trace/ultratrace minerals may be included such Iron (0.27 to 27 mg), Zinc (2 to 13 mg), Copper (200 to 1300 μgrams), Selenium (15 to 70 μgrams), Chromium (0.2 to 45 μgrams), Iodine (110 to 290 μgrams), Manganese (0.003 to 2.6 mg), and Molybdenum (2 to 50 μgrams).

In a further example a gummy matrix may be formed. In one such embodiment, the gummy matrix is a mixed gel. In a further embodiment, the mixed gel may comprise 7% to 9% of gelatin or gelatin in combination with gellan gum, pectin, modified starch, fructo-oligosaccharide, and hydrocolloid source(s), by weight.

In a further embodiment, sugars may be added to the gummy matrix. When sugar is added the gummy matrix may consists of 5.0% to 8.0% of sugar. Artificial sweetener(s) may be used instead, or in addition to sugar, at 22.0% to 25.0%, by weight. When sweetener(s) are added the gummy matrix may consists of 7.0% to 9.0% of gelatin or gelatin in combination with gellan gum, pectin, modified starch, fructo-oligosaccharide and hydrocolloid source(s), by weight.

In a further embodiment, protein sources may be added to the gummy matrix. When protein is added the gummy matrix may consists of 14.0% to 22.0% of bioactive protein. When the protein source(s) is added the gummy matrix may consists of 7.0% to 9.0% of gelatin or gelatin in combination with gellan gum, pectin, modified starch, fruct-ooligosaccharides and hydrocolloids, by weight.

In a further embodiment, an amino acid and/or amino acid metabolite source(s) may be added to the gummy matrix. When an amino acid source is added the gummy matrix may consists of 3.0% to 9.0% of bioactive amino acid and/or amino acid metabolite source(s). When the protein source is added the gummy matrix may consists of 7.0% to 9.0% of gelatin or gelatin in combination with gellan gum, pectin, modified starch, fructooligosaccharides and hydrocolloids, by weight.

In a further embodiment, a lipid source(s) may be added to the gummy matrix. When a lipid source is added the gummy matrix may consists of 3.0% to 9.0% of lipid(s) comprising of omega 3 and omega 6 fatty acids, by weight. When the lipid source is added the gummy matrix may consists of 7.0% to 9.0% of gelatin or gelatin in combination with gellan gum, pectin, modified starch, fructooligosaccharides and hydrocolloids, by weight.

In a further embodiment, an antioxidant/anti-carcinogenic source(s) may be added to the gummy matrix. When an antioxidant/anti-carcinogenic source(s) is added the gummy matrix may consists of 0.2% to 0.8% of, by weight. When the antioxidant/anti-carcinogenic source is added the gummy matrix may consists of 7.0% to 9.0% of gelatin or gelatin in combination with gellan gum, pectin, modified starch, fructooligosaccharides and hydrocolloids, by weight.

During processing, the mixing temperature should be raised to, at least, a range between 55° C. to 70° C., and then gelatin should be added. The gelatin should be stirred until it is completely dissolved, standing at the same temperature for a range of 15 minutes to 3.5 hours. Optimal gel strength as a function of gel concentration may range from 100 to 200 bloom at 10° C. Other possible ingredients (e.g., gellan gum and pectin with cane or beet sugars or the listed artificial sweeteners (e.g., sugar alcohols) may be added in an amount 0.1 to 5 times the weight of the solution. The mixture is slow boiled until the final solution is ready.

Once the gummy matrix is ready, sugars can be added following a melting phase in water-dispersed, modified starch paste in an amount at least twice the weight thereof. The sugar solution should boil until the solution reaches 70 to 81 Brix as measured by Brix spindle and a vacuum degassing should be performed after complete mixing of ingredients so the solution contains no bubbles. The mixed solution should be 79.9° C. to 96° C. and cooled to 40 to 45° C. The active ergogenic ingredients may be mixed and blended at this point and poured into a blending barrel. Other ingredients, such as flavor agents, such as sour agents, edible essence, edible colorant, etc., may be added to the mixed solution. The solution should then be homogenously stirred and blended and the mixed gummy solution should next be casted/molded by transferring the blended solution into a casting machine.

Additional ingredients, including but not limited to the following may also be added to the gummy matrix: beta-glucans, dextrans, dextrins/maltodextrins, guar, locust bean, plant extracts (Arabic and tragacanth), carrageenan (seaweed extract, eucheuma cottonii, eucheuma spinosum, gigartina species), Irish Moss, Eucheuma species), brand sta-slim (supplier A. E. Staley, modified potato starch), brand stellar (supplier A. E. Staley, acid modified corn starch), brand Amalean 1 (supplier Am. Maize, modified high amylose corn starch), brand Paselli SA2 (supplier Avebe America Inc., potato maltodextrin), brand oatrim (supplier conagra, Quaker Oats, oat maltodextrin), brand novagel (supplier FMC, microcrystalline cellulose), brand maltrin (supplier grane processing, corn maltodextrin), brand splendid (supplier Hercules, Inc, citrus pectin), brand n-oil (supplier Nat. Starch, tapioca dextrin), brand litesse (supplier Pfizer, polydextrose), brand ricetrinthree (3) (supplier Zumbro, rice maltodextrin).

In a further embodiment, the protein source is a gummy product that may employ micro-encapsulation technology for extended release of important synergistic ingredients infused within a gummy dosage form. In one embodiment, beta-hydroxy-beta-methylbutyrate (“HMB”) microspheres may be employed.

Microencapsulation is the process of enclosing these ingredients into a cell by coating them with a proper substance in a way that results in appropriate cell release in the intestinal medium. For purposes of example only and not intended to be limiting, HMB and KIC microspheres may be prepared through an ionic gelation process using sodium alginate as a polymer. For purposes of example only and not intended to be limiting, HMB microspheres were prepared through an ionic gelation process using sodium alginate as a polymer. See FIG. 3. The sodium alginate polymer was dispersed in a required amount of distilled water to form a homogenous polymer mixture. The amount of water in the gummy matrix may be determined by adding twice the amount of water to the weight of the prepared gelatin. In a further embodiment, water may be added in a range from 5 to 12 times the weight of the prepared gelatin. In a further embodiment, water should be at least 10% to 20% of the gelatin weight. In a more preferred range, water may be 14% to 18% of total prepared gelatin weight. HMB was added to the polymer dispersion and mixed thoroughly with a magnetic stirrer to form a viscous homogenous dispersion. The resulting dispersion was then added manually drop wise into calcium chloride (10% w/v) solution (40 ml) through a syringe with a needle of size no. 20. The added droplets were retained in the calcium chloride solution for 30 minutes to complete the curing reaction and to produce spherical rigid microcapsules having a different coat:core ratio (1:1).

It is possible that time-release KIC and HMB microcapsules may have incomplete forms. By increasing the polyelectrolyte complex with oppositely charged polyelectrolytes, however, the mechanical strength of the hydrogel and/or permeability barrier may be enhanced. For example, addition of a polycation (such as but not limited to poly-L-lysine) allows a membrane of polyelectrolyte complex to form on the surface of alginate beads.

In further preferred embodiment, the protein source may include artificial sweeteners such as D-sorbitol, D-mannitol, erythritol, xylitol, thaumatins, glycyrrhizin, stevioside, tagatose, acesulfame potassium, alitame, saccharin, dextrose, neotame, acesulfame K (Aka Sunette, Sweet One, Sweet n Safe Acesulfame-k), citrus pectin, tapioca dextrin, polydextrose, rice maltodextrin, nutrasweet (brand simplesse), nutrasweet (brand simplesse 100), national starch (brand n-flate), Nabisco (brand salatrim), proctor & Gamble (brand caprenin), Proctor & Gamble (brand Olestra), aspartame, stevie, sucralose, maltodextrose, sucrose, high fructose corn syrup. In one embodiment, the amount of sweetener may range from 1 to 50 grams per batch and may be scaled up to 50,000 kilograms for manufacturers. In a preferred embodiment, the amount of sweetener may range from 1 to 10 grams per batch.

The protein source may also include whole proteins. Suitable whole proteins include but are not limited to whey protein isolate, soy protein, casein protein, egg protein, beef protein, and hemp protein. The amount of whole protein may range from 1 to 10 grams. The protein source may also include Branched Chain Amino Acids. These include but are not limited to L-Leucine, L-Isoleucine, and L-Valine. In one embodiment, the branched chain amino acids may be present from 1 to 50 grams per batch and scaled up to 50,000 kilograms for manufacturers. In a preferred embodiment, the branched chain amino acids may be present from 1 to 8 grams. The protein source may also include L-alanyl-L-glutamine that may be present from 1 to 50 grams per serving and scaled up to 50,000 kilograms for manufacturers. In a preferred embodiment, L-alanyl-L-glutamine may be present from 1 to 8 grams per serving. This embodiment may also include essential amino acids including but not limited to L-Histidine, L-Lysine, L-Methionine, L-Phenylalanine, and L-Threonine. These may be present from 1 to 50 grams per serving and scaled up to 50,000 kilograms for manufactures. In a preferred embodiment, they are present from 1 to 10 grams. This embodiment may also include L-Arginine from 1 to 50 grams per serving and scaled up to 50,000 kilograms for manufactures. In a preferred embodiment, L-Arginine is present from 1 to 10 grams. Trans-Resveratrol from 1 to 50 grams per serving and scaled up to 50,000 kilograms for manufactures. In a preferred embodiment, Trans-Resveratrol is present from 1 to 10 grams. Microencapsulated HMB may be present from 1 to 50 grams per serving and scaled up to 50,000 kilograms for manufactures. In a preferred embodiment, Microencapsulated HMB is present from 1 to 10 grams. This embodiment may also include a Gummy Solution. The Gummy solution may include but is not limited to gelatin, potassium, sodium, chloride, magnesium, citric acid present at 85%, by weight.

A further embodiment provides mixing branched chain amino acids and trans-resveratrol for at least 15 to 45 minutes, preferably from 15 to 30 minutes, while cooling temperatures are below 150° C. and closer to 55° C. This method includes mixing whole proteins for less than 15 minutes after gummy matrix temperature is reduced from 60.25° C. and closer to 40 to 45° C. The resulting gummy matrix may be stored at 15° C. to 29.9° C. at a relative humidity of 10% to 41%, and drying the product to a water content of 8% to 21% or water activity at 0.65 to 0.75. As used herein, “%” refers to “% by mass”, unless otherwise specified. This embodiment may also include adding microencapsulated HMB during the cooling phase. Cinnamon extract flavoring may be used to enhance flavor.

Also included in this disclosure is a method of making an improved protein source. The method includes preparing whole proteins at a temperature range of 90 to 113° F. for less than 15:00 minutes. This embodiment may also include preparing branched chain amino Acids, L-alanyl-L-glutamine, and essential amino acids, L-Arginine, and Trans-Resveratrol: 100 to 284 degrees Fahrenheit for less than 30 minutes. This embodiment may also include adding microencapsulated HMB during the cooling phase. In a further embodiment, the branched chain amino acids, L-alanyl-L-glutamine, essential amino acids, L-Arginine, Trans-Resveratrol may be combined in the stirring chamber prior to adding the whole proteins. Microencapsulated HMB may then be added during the cooling phase.

In another embodiment, a protein delivery system may be made from two portions of distilled water, in combination with 0.1% to 0.5% citric acid mixed with 3.0% to 10.0% hydrocolloid, 1.0% to 25% reducing or non-reducing sweetener, 5.0% to 45.0% liquid sugar, 1.0% to 10.0% artificial sweetener (e.g., polyols, alcohol sugars) 0.01% to 1.5% food additive (e.g., sodium lactate), 0.001% to 0.15% preservative, 0.001% to 0.50% color, and 0.001% to 0.50% flavor. Oven dry methods, as known to those of skill in the art, for 10-18 hrs at 45-75 C may be used with this embodiment.

In a preferred embodiment reducing sugars (e.g., glucose, dextrose, fructose, etc.) may not be not included. For example, research has shown that these monosaccharides can form cross bridges with certain amino acids in whey protein, which degrades the high quality amino acid sequence.

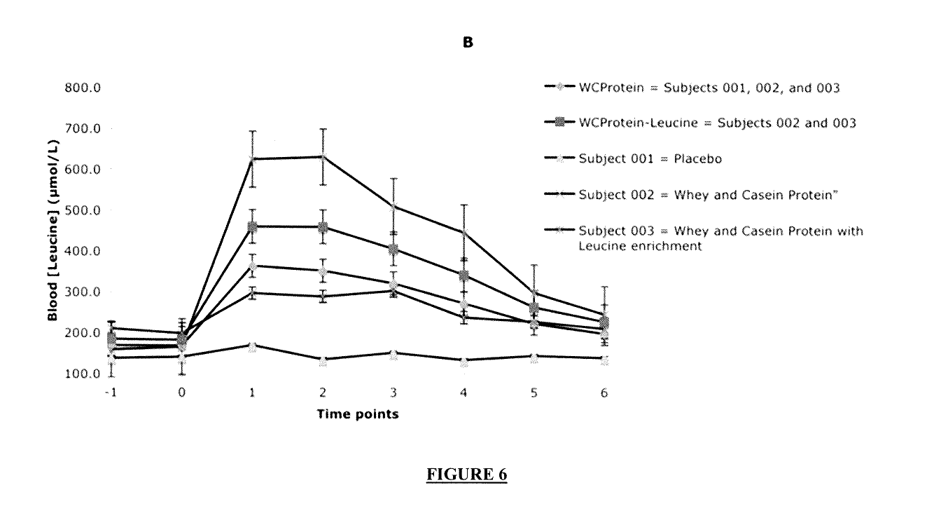

For the present invention, a single site, single-blind, randomized, placebo-controlled, pilot trial conducted in males who ingested a semi-solid gummy matrix embodiment containing 5.0 grams of whey protein isolate and 5.3 grams of micellar casein protein (n=3) resulted in non-significant (p>0.05) increases by 116.3% in blood leucine concentrations. In addition, another group (n=2) who ingested 6.6 grams of whey protein isolate and 2.1 grams of micellar casein protein that was combined with 3.3 grams of leucine also resulted in non-significant increases by 153.1% in blood leucine concentrations. It is likely that these changes were non-significant due to small heterogeneous sample sizes. See FIG. 6.

PILOT TEST—A Single Site, Single-Blind, Randomized, Placebo-Controlled, Pilot Trial to Evaluate the Potential Effects of a Dietary Protein Delivery System on Nutrient Absorption and Metabolism in Males. Name of Main Active Ingredients: Whey protein isolate, micellar casein protein, 1-leucine, canola/olive oil blend, ascorbic acid, and Sus scrofa domesticus gelatin. The primary objectives of the test were to assess the effects of a dietary protein delivery system on nutrient absorption and metabolism as compared to placebo. A secondary objective was to collect information on the safety, tolerability and adverse effects and subjective experiences related to the use of this protein delivery system by study subjects.

Protocol: This was a single site, one-visit, single blind, randomized, placebo-controlled, pilot trial to evaluate the potential effects of two dietary protein delivery systems on nutrient absorption in males. Subjects (n=3) were told to refrain from physical exercise for 72 h prior to the experimental trial and to consume their normal evening meal no later than 22.00 h. Therefore, all subjects reported to the lab in an overnight postabsorptive state. On the morning of the experiment at ˜08.30 h all subjects signed a written informed consent form, and were oriented to the testing procedures. Eligible subjects were randomized and combined into two separate treatments (supplements=whey and casein protein combined with a placebo [WC_{PROTEIN}; n=3] or whey and casein protein with leucine enrichment [WC_{PROTEIN-LEUCINE}; n=2], See FIGS. 6-13). Each subject was administered one treatment immediately after completing the second blood draw at baseline 2. Subjects consumed 100 mL of drinking water hourly.

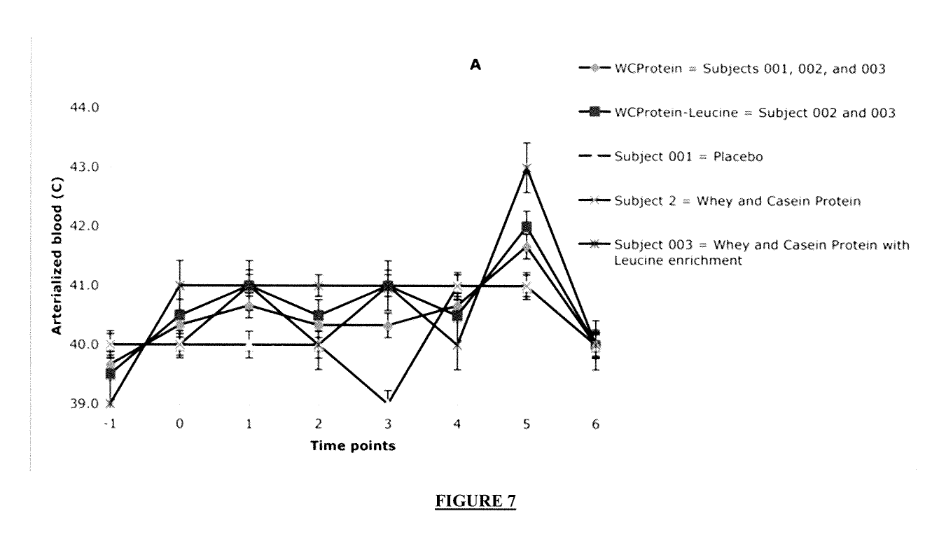

FIG. 7 shows arterialized blood temperature measures. Data presented are means±standard error of the mean for WC_{PROTEIN} and WC_{PROTEIN-LEUCINE} groups. Individual subjects data is also presented. There were no significant differences (p>0.05) among baseline 1, baseline 2, and all post-ingestion time-points for arterialized blood temperature.

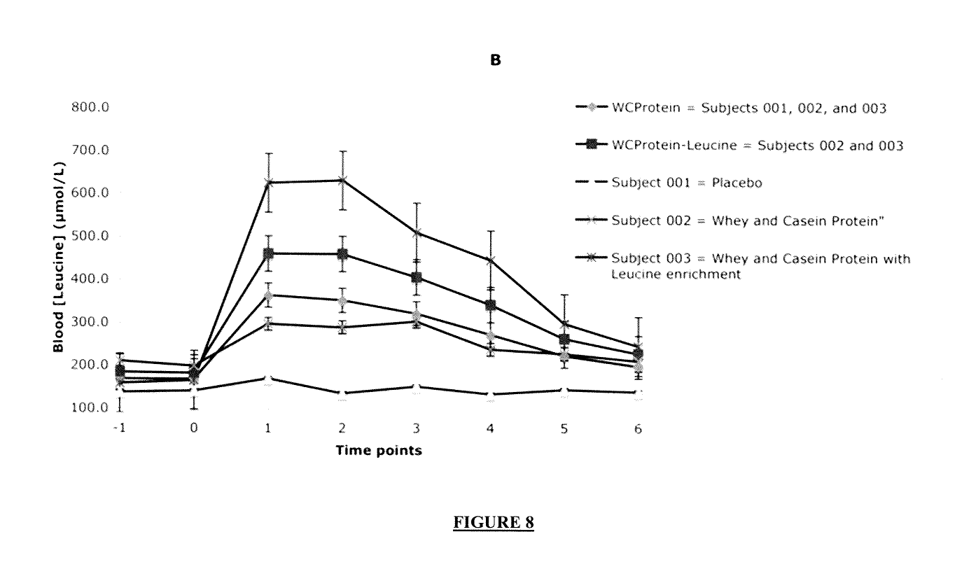

FIG. 8 shows leucine blood measures. Data presented are means±standard error of the mean for WC_{PROTEIN} and WC_{PROTEIN-LEUCINE} groups Individual subjects data is also presented. For both groups there were no significant differences (p>0.05) in leucine concentrations among baseline 1, baseline 2, and all post-ingestion time-points.

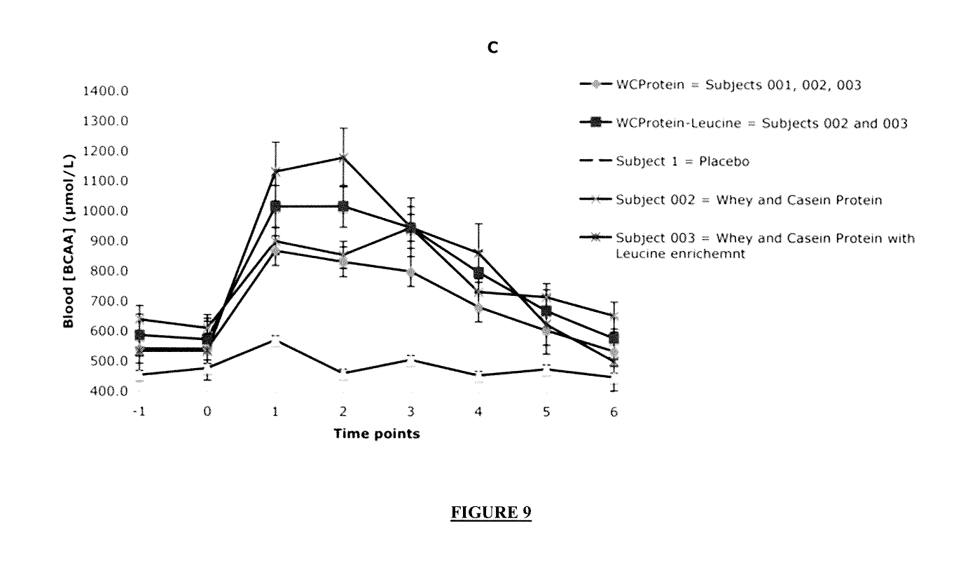

FIG. 9 shows branched chain amino acid blood measures. Data presented are means±standard error of the mean for WC_{PROTEIN} and WC_{PROTEIN-LEUCINE} groups. Individual subjects data is also presented. For the WC_{PROTEIN} group there were no significant differences (p≦0.05) between time-points −1 (baseline 1) and 0 (baseline 2); but time-point 4 was 21.3% greater than time-point 1. In the WC_{PROTEIN-LEUCINE} group there were no significant differences (p>0.05) between time-points −1 and 0; but time-point 5 was 14.2% and 17.0% greater than time-points −1 and 0, respectively (p≦0.05).

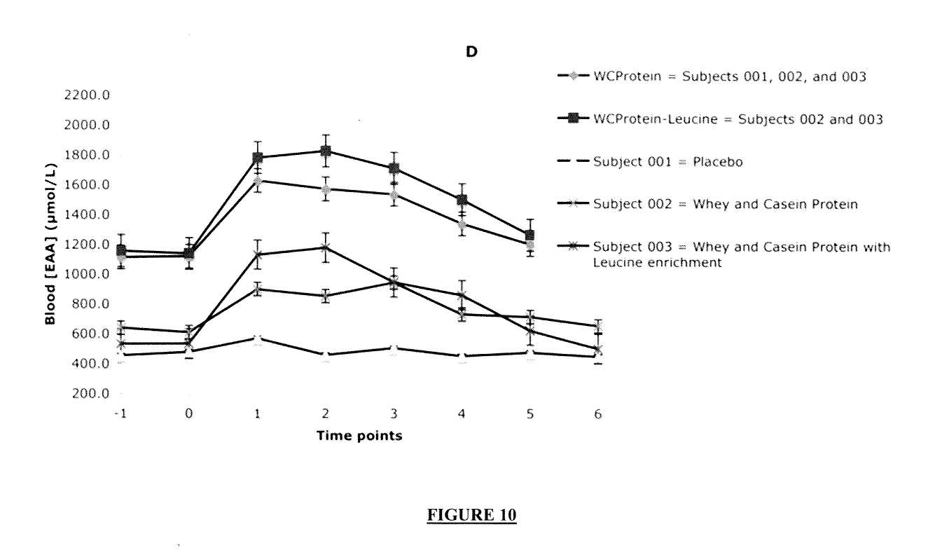

FIG. 10 shows essential amino acid blood measures. Data presented are means±standard error of the mean for WC_{PROTEIN} and WC_{PROTEIN-LEUCINE} groups. Individual subjects data is also presented. For the WC_{PROTEIN} group there were no significant differences (p>0.05) between time-points −1 (baseline 1) and 0 (baseline 2); but time-point 5 was 8.1% greater than time-points −1 (p≦0.05). Time-point 4 was 17.7% greater than time-point 1 (p≦0.05). In the WC_{PROTEIN-LEUCINE} group there were no significant differences between time-points −1 and 0 (p>0.05); but time-point 0 was 11.2% greater than time-point 5 and time-point 1 was 15.7% greater than time-point 4 (p≦0.05).

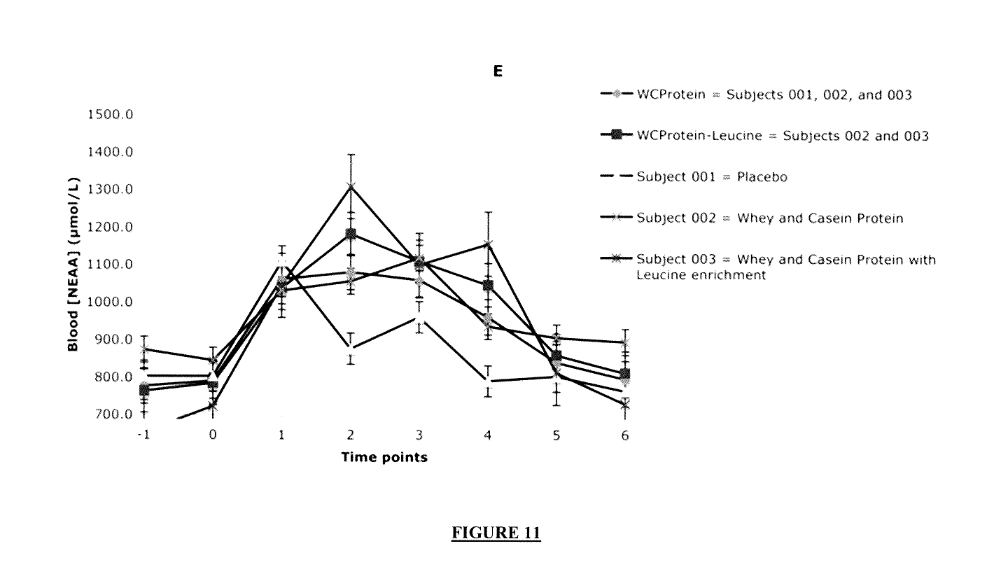

FIG. 11 shows non-essential amino acid blood measures. Data presented are means±standard error of the mean for WC_{PROTEIN} and WC_{PROTEIN-LEUCINE} groups. Individual subjects data is also presented. For the WC_{PROTEIN} group there were no significant differences (p>0.05) between time-points −1 (baseline 1) and 0 (baseline 2); but time-point −1 was 36.6% greater than time-point 1 (p≦0.05); time-point 0 was 34.3% and 34.1% greater than time-point 1 and time-point 3, respectively (p≦0.05); time-point 1 was 20.8% and 25.0% greater than time-point 5 and time-point 6, respectively (p 0.05); time-point 2 was 11.1% greater than time-point 4 (p≦0.05); time-point 3 was 20.7% and 24.9% greater than time-point 5 and time-point 6, respectively (p≦0.05). In the WC_{PROTEIN-LEUCINE} group there were no significant differences (p>0.05) between time-points −1 (baseline 1) and 0 (baseline 2) and only a main effect for time (collapsed across blood concentrations, p=0.059) was detected (p<0.05).

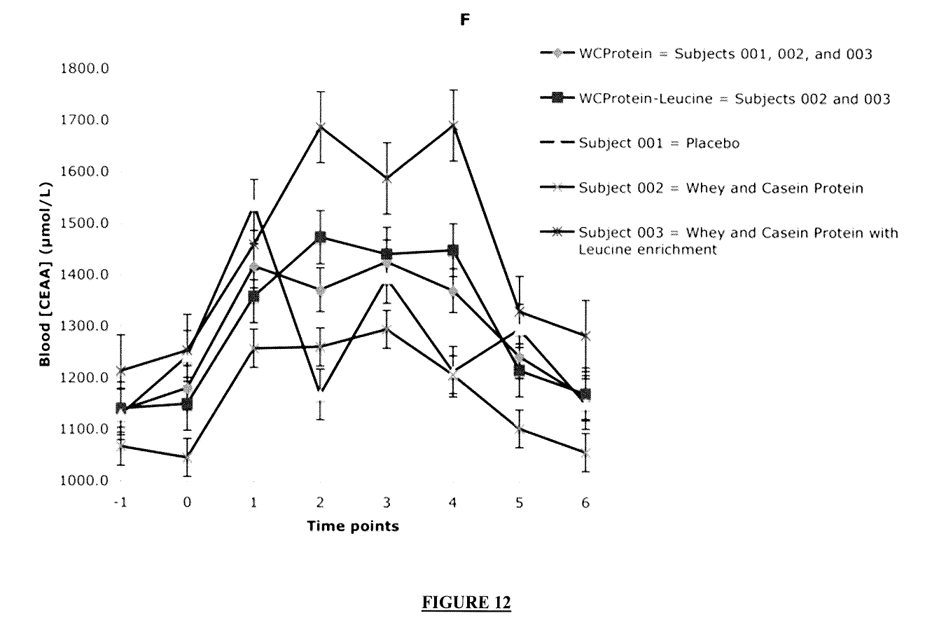

FIG. 12 shows conditionally essential amino acid blood measures. Data presented are means±standard error of the mean for WC_{PROTEIN} and WC_{PROTEIN-LEUCINE} groups. Individual subjects data is also presented. For the WC_{PROTEIN} group there were no significant differences (p>0.05) between time-points −1 (baseline 1) and 0 (baseline 2); but time-point −1 was 24.8% and 25.5% greater than time-point 1 and time-point 3, respectively (p≦0.05); time-point 0 was 20.1%, 20.8%, and 5.2% greater than time-point 1, time-point 3, and time-point 5, respectively (p≦0.05); time-point 1 was 12.4% greater than time-point 5 (p<0.05); time-point 3 was 12.9% and 18.5% greater than time-point 5 and time-point 6, respectively (p≦0.05). In the WC_{PROTEIN-LEUCINE} group there were no significant differences (p>0.05) between time-points −1 (baseline 1) and 0 (baseline 2); but time-point 0 was 18.2% greater than time-point 1 (p≦0.05); time-point 1 was 10.5% and 91.6% greater than time-point 5 and time-point 6, respectively (p≦0.05). Time-point 5 was 3.8% greater than time-point 6 (p<0.05).

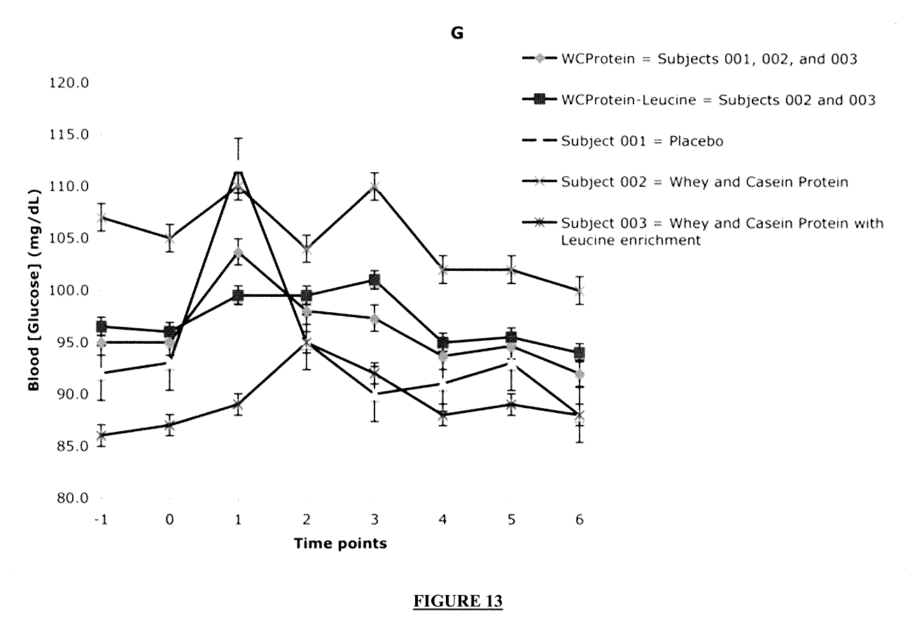

FIG. 13 shows glucose blood measures. Data presented are means±standard error of the mean for WC_{PROTEIN} and WC_{PROTEIN-LEUCINE} groups. Individual subjects data is also presented. For both groups there were no significant differences (p>0.05) in glucose concentrations among time-point −1 (baseline 1), 0 (baseline 2), and all post-ingestion time-points (1-6).

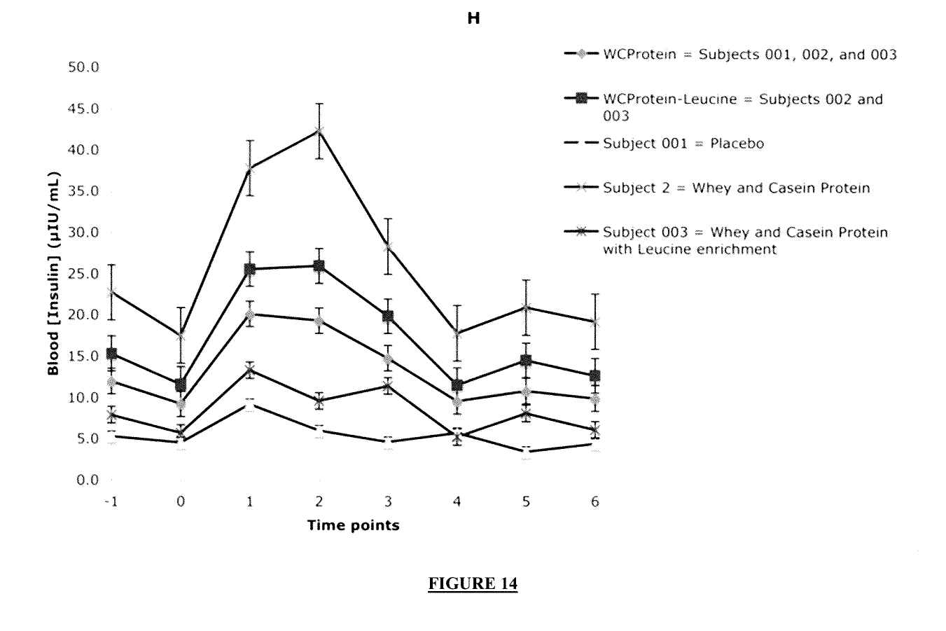

FIG. 14 shows insulin blood measures. Data presented are means±standard error of the mean for WC_{PROTEIN} and WC_{PROTEIN-LEUCINE} groups. Individual subjects data is also presented. For both groups there were no significant differences (p>0.05) in insulin concentrations among time-points −1 (baseline 1), 0 (baseline 2), and all post-ingestion time-points (1-6).

For this repeated measures experimental design, subjects had repeated blood samples taken from the arm for two baseline measures separated by ˜1 h (time-point 1 [Baseline 1]=−1 {0955-0959 EST} and time-point 2 [Baseline 2]=0 {1034 EST}). In addition, six blood samples each separated by ˜0.5 were taken over ˜3 h period after baseline 2 (See FIG. 13).

Arterialized blood samples (>37.0 C) were taken with sanitized 23.75 gauge needles from the antecubital vein over the course of the experimental trial. Arterialized blood samples were obtained repeatedly over the course of the experiment by wrapping a temperature controlled heating blanket around the forearm. This method of blood sampling has been reported to serve as a suitable surrogate for direct arterial sampling in whole body studies. An infrared thermometer was used to determine the skin temperature. Blood samples for all profiles were centrifuged at 3320 RPM for 10 minutes and 0.10 mL of cells were separated and stored in a refrigerator at 22 C. For the amino acid specimens, cells were frozen at 3.0 C until further analyses. For glucose and insulin, the specimen sat for 10 minutes to clot before being spun and stored in a refrigerator at 22 C until further analyses.

Reliability

Blood samples were taken for evaluation of Serum: Alanine aminotransferase (ALT/SGPT); albumin:globulin (A-G) ratio; albumin, serum; alkaline phosphatase, serum; aspartate aminotransferase (AST/SGOT); bilirubin, total; BUN; BUN:creatinine ratio; calcium, serum; carbon dioxide, total; chloride, serum; creatinine, serum; eGFR calculation; globulin, total; glucose, serum; insulin, serum; creatine kinase, serum; potassium, serum; protein, total, serum; sodium, serum, and Plasma: :α-alanine; β-Alanine; Alloisoleucine; α-Aminoadipic acid; γ-Aminobutyric acid (GABA); β-Aminoisobutyric acid; α-Amino-N-butyric acid; Arginine; Argininosuccinic acid; Asparagine; Aspartic acid; Citrulline; Cystathionine; Cystine; Glutamic acid; Glutamine; Glycine; Histidine; Homocitrulline; Homocystine; Hydroxylysine; Hydroxyproline; Isoleucine; Leucine; Lysine; Methionine; Ornithine; Phenylalanine; Proline; Sarcosine; Serine; Taurine; Threonine; Tryptophan; Tyrosine; Valine. Additionally, routine clinical laboratory tests (hematology and chemistry) were evaluated. Throughout the study subjects were asked to subjectively rate the level of tolerability and adverse effects and subjective experiences related to the use of the protein delivery system.

The results of the pre-ingestion baseline measurements (baseline 1 and baseline 2) for the two groups (WC_{PROTEIN} and WC_{PROTEIN-LEUCINE}) were used to determine the test-retest reliability for arterialized blood temperature and all blood measurements. There were no significant (p>0.05) differences between mean values for test vs. retest for all variables, and therefore, each group served as its own control.

Statistical Analyses

Eight separate one-way repeated measures ANOVAs were used to analyze arterialized blood temperature and blood concentrations of leucine, branched chain amino acids (BCAA=leucine, isoleucine, and valine), essential amino acids (EEA=isoleucine, leucine, lysine, methionine, phenylalanine, threonine, tryptophan, and valine), non-essential amino acids (NEEA=alanine, asparagine, aspartate, citrulline, glutamate, glycine, and serine), conditional amino acids (CEAA=arginine, cystine, glutamine, histidine, proline, taurine, and tyrosine), glucose, and insulin. Follow-up analyses included paired t-tests. An alpha level of p≦0.05 was considered statistically significant for all ANOVA analyses. SPSS (version 22; SPSS, Inc., Chicago, Ill.) was used for all statistical analyses.

Results

Arterialized Blood Temperature

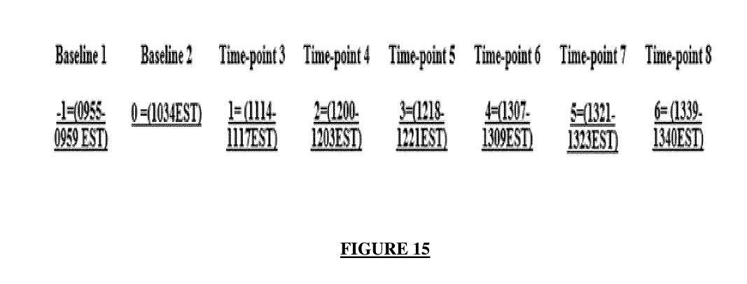

FIG. 15, shows the mean (±standard error of the mean [SEM]) values for baseline 1, baseline 2, and all time-points for arterialized blood temperatures and blood concentrations of leucine, branched chain amino acids, essential amino acids, non-essential amino acids, conditional amino acids, glucose, and insulin. For both groups there were no significant differences among baseline 1, baseline 2, and all post-ingestion time-points (1114-1340 EST) for arterialized blood temperature.

Leucine

For both groups there were no significant differences in leucine concentrations among baseline 1, baseline 2, and all post-ingestion time-points.

Branched Chain Amino Acids

For the branched chain amino acid concentrations in the WC_{PROTEIN} group there were no significant differences between baseline 1 and baseline 2; but time-point 6 (1307-1309 EST) was 21.3% greater than time-point 3 (1114-1117 EST). In the WC_{PROTEIN-LEUCINE} group there were no significant differences between baseline 1 and baseline 2; but time-point 7 (1321-1323 EST) was 14.2% and 17.0% greater than and baseline 1 and baseline 2, respectively.

Essential Amino Acids

For the essential amino acid concentrations in the WC_{PROTEIN} group there were no significant differences between baseline 1 and baseline 2; but time-point 7 was 8.1% greater than baseline 1 and time-point 6 (1307-1309 EST) was 17.7% greater than time-point 3. In the WC_{PROTEIN-LEUCINE} group there were no significant differences between baseline 1 and baseline 2; but baseline 2 was 11.2% greater than time-point 7 and time-point 3 was 15.7% greater than time-point 6.

Non-Essential Amino Acids

For the non-essential amino acid concentrations in the WC_{PROTEIN} group there were no significant differences between baseline 1 and baseline 2; but baseline 1 was 36.6% greater than time-point 3; baseline 2 was 34.3% and 34.1% greater than time-point 3 and time-point 5 (1218-1221 EST), respectively; time-point 3 was 20.8% and 25.0% greater than time-point 7 and time-point 8 (1339-1340 EST), respectively; time-point 4 (1200-1203 EST) was 11.1% greater than time-point 6; time-point 5 was 20.7% and 24.9% greater than time-point 7 and time-point 8, respectively. In the WC_{PROTEIN}-LEUCINE group there were no significant differences between baseline 1 and baseline 2 concentrations and only a main effect for time (collapsed across blood concentrations, p=0.059) was detected, which was likely to a small heterogeneous sample size.

Conditionally Essential Amino Acids

For the conditionally essential amino acid concentrations in WC_{PROTEIN} group there were no significant differences between baseline 1 and baseline 2; but baseline 1 was 24.8% and 25.5% greater than time-point 3 and time-point 5, respectively; baseline 2 was 20.1%, 20.8%, and 5.2% greater than time-point 3, time-point 5, and time-point 7, respectively; time-point 3 was 12.4% greater than time-point 7; time-point 5 was 12.9% and 18.5% greater than time-point 7 and time-point 8, respectively. In the WC_{PROTEIN-LEUCINE} group there were no significant differences between baseline 1 and baseline 2 concentrations; but baseline 2 was 18.2% greater than time-point 3; time-point 3 was 10.5% and 91.6% greater than time-point 7 and time-point 8, respectively. Time-point 7 was 3.8% greater than time-point 8.

Glucose

For both groups there were no significant differences in glucose concentrations among baseline 1, baseline 2, and all post-ingestion time-points.

Insulin

For both groups there were no significant differences in insulin concentrations among baseline 1, baseline 2, and all post-ingestion time-points.

### SUMMARY

The results of the present study indicate that both WC_{PROTEIN} and WC_{PROTEIN-LEUCINE} groups acted as their own control group. The present invention resulted in significant changes in blood concentrations of BCAAs. For the WC_{PROTEIN} group there was a 21.3% increase in blood concentrations and 14.2% to 17.0% increases for the WC_{PROTEIN-LEUCINE} group. Thus, for both groups the present invention increased the concentrations of BCAAs. It is well documented that BCAAs when combined with resistance training and proper diet will increase and/or maintain muscle mass.

The EAA concentrations from baseline 1 and baseline 2 increased by 8.1% to 11.2% at time-point 7 in the WC_{PROTEIN} group and WC_{PROTEIN-LEUCINE} group, respectively. In addition, for the WC_{PROTEIN} group, time-point 6 was 17.7% greater than time-point 3. In contrast, for the WC_{PROTEIN-LEUCINE} group, there was a 15.7% decrease in EAAs from time-point 3 to time-point 6 (See Table X). Thus, with or without the influence of a placebo, the EAAs remained elevated for approximately two hours.

From baseline 1 and baseline 2 the NEAAs in the WC_{PROTEIN} group increased at time-point 3 and time-point 5 by 34.1% to 36.6%. In addition, time-point 3 was 20.8% greater than time-point 7 and 25.0% greater than time-point 8, along with time-point 5 being 20.7% greater than time-point 7 and 24.9% greater than time-point 8. Furthermore, time-point 4 was 11.1% greater than time-point 6. For the WC_{PROTEIN-LEUCINE} group, the study only detected a significant main effect for time and no significant differences among the post-hoc comparisons. Again, this was likely due to a smaller heterogeneous sample size. It is well known that NEAA are synthesized endogenously, and therefore, both groups provided the necessary nutrient absorption. For example, conditionally essential amino acids (CEAA) can be considered to be essential based on the body's inability to actually synthesize them from other amino acids under certain conditions. In the present study the CEAA concentrations increased for both groups.

For the CEAAs from baseline 1 and baseline 2 in the WC_{PROTEIN} group there were 20.8% to 25.5% increases at time-point 3 and time-point 5. In addition, baseline 2 was 5.2% greater than time-point 7, time-point 3 was 12.4% greater than time-point 7, and time-point 5 was 12.9% to 18.5% greater than time-point 7 and time-point 8. In the WC_{PROTEIN-LEUCINE} group baseline 2 was 18.2% greater than time-point 3 and time-point 3 was 10.5% and 91.6% greater than time-point 7 and time-point 8, respectively. In addition, time-point 7 was 3.8% greater than time-point 8.

Although there were no changes in blood glucose or insulin for both groups, FIG. 14 portrays similar patterns of insulin concentrations. Insulin acts at the skeletal muscle for glucose uptake and thus promotes amino acid utilization. Thus, the present invention appears to induce insulin secretion. This pattern of response was likely non-significant due to a small heterogeneous sample sizes.

In summary, both groups using the present inventions delivery system, avoided large amounts of protein degradation during processing and resulted in sufficient amino acid absorption rates. Future studies should further examine nutrient absorption as well as examine the effects of the present invention on human performance in males and females.

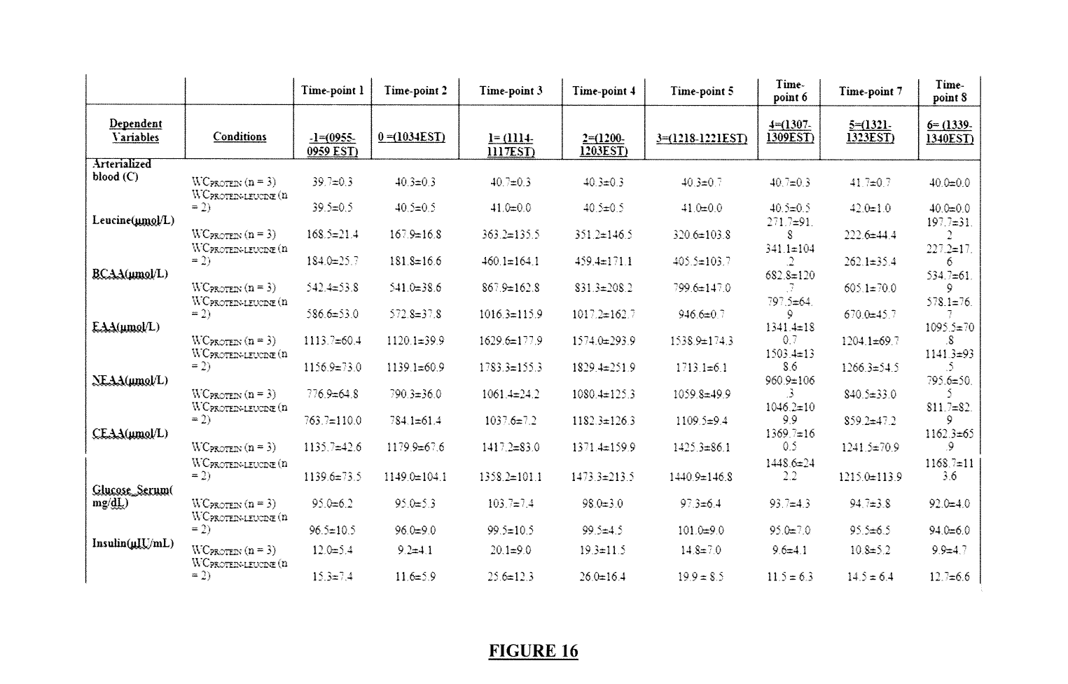

FIG. 16 shows arterialized blood values and blood concentrations (mean±SEM) of leucine, branched chain amino acids (BCAA), essential amino acids (EAA), non-essential amino acids (NEAA), conditional amino acids (CEAA), glucose, and insulin at pre-ingestion 1 (0955-0959 EST), pre-ingestion 2 (1034 EST), and at each post-ingestion time-point (1114-1340 EST). There were significant (p<0.05) changes in blood concentrations in WC_{PROTEIN} group (n=3) for BCAA from 1114-1117 EST to 1307-1309 EST; EEA from 0955-0959 EST to 1321-1323 EST and 1114-1117 EST to 1307-1309 EST; NEAA from 0955-0959 EST to 1114-1117 EST, 1034 EST to 1114-1117 EST, 1034 EST to 1218-1221 EST, 1114-1117 EST to 1321-1323 EST, 1114-1117 EST to 1339-1340 EST, 1200-1203 EST to 1307-1309 EST, 1218-1221 EST to 1321-1323 EST, 1218-1221 EST to 1339-1340 EST; and CEAA from 0955-0959 EST to 1114-1117 EST, 0955-0959 EST to 1218-1221 EST, 1034 EST to 1114-1117 EST, 1034 EST to 1218-1221 EST, 1034 EST to 1321-1323 EST, 1114-1117 EST to 1321-1323 EST, 1218-1221 EST to 1321-1323 EST, and 1218-1221 EST to 1339-1340 EST. For the WCPROTEIN-LEUCINE group (n=2), there were significant changes in blood concentrations for BCAA from 0955-0959 EST to 1321-1323 EST; EAA from 1034 EST to 1321-1323 EST and 1114-1117 EST to 1307-1309 EST; a significant main effect (p=0.59) for NEAA; CEAA from 1034 EST to 1114-1117 EST, 1114-1117 EST to 1321-1323 EST, 1114-1117 EST to 1339-1340 EST, and 1321-1323 EST to 1339-1340 EST. The follow-up t-tests of the group means for all dependent variables indicated that there was no significant difference (p>0.05) between pre-ingestion 1 (time point=1) and pre-ingestion 2 (time point=2).

The current disclosure promotes MPS, attenuates muscle protein degradation, and increases insulin blood levels in healthy individuals, improves muscular endurance at a high rate. The current disclosure may function synergistically with trans-resveratrol for decreasing the levels of cellular oxidative stress or cell damage from free radical formation. The current disclosure may also promote anti-aging as well as may protect from cancer cell proliferation. Moreover, the current disclosure provides a convenient delivery system that may be in the form of a gummy candy, as opposed to the drinks and powders used by others. The current disclosure may also be suitable for individuals with type 2 diabetes, or who are overweight and obese. Typically, these individuals are recommended to low glycemic diets with caloric restriction. In part, these designs, limit the consumption of foods with high amounts of table sugar or sucrose.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A protein delivery system comprising:
at least one protein;
at least one essential amino acid;
at least one caloric intake reducing agent;
at least one anti-carcinogenic factor; and
wherein the protein delivery system is a semi-solid.

2. The protein delivery system of claim 1, wherein the at least one protein is present from 14 to 20 percent by weight.

3. The protein delivery system of claim 1, wherein the at least one essential amino acid is present from 3 to 9 percent by weight.

4. The protein delivery system of claim 1, wherein the at least one anti-carcinogenic factor is present from 0.2 to 0.8 percent by weight.

5. A protein delivery system comprising:
at least one protein source;
at least one amino acid source;
at least one amino acid metabolite source;
at least one enzyme source;
at least one lipid source;
at least one vitamin;
at least one mineral;
at least one botanical/herbal-extract;
at least one caloric intake reducing agent;
at least one anti-carcinogenic factor; and
wherein the protein delivery system is a semi-solid.

6. The protein delivery system of claim 5, wherein the protein source is present from 14 to 20 percent by weight.

7. The protein delivery system of claim 5, wherein the amino acid source is present from 3 to 9 percent by weight.

8. The protein delivery system of claim 5, wherein the lipid source is present from 3 to 9 percent by weight.

9. The protein delivery system of claim 8, wherein the lipid sources comprises omega 3 and omega 6 fatty acids.

10. The protein delivery system of claim 5, wherein the anti-carcinogenic factor is present from 0.2 to 0.8 percent by weight.

11. The protein delivery system of claim 5, wherein the vitamin source is present from 0.2 to 0.8 percent by weight.

12. A method of forming a gummy protein delivery device comprising:
forming a gummy matrix by integrating ingredients;
heating the gummy matrix;
adding gelatin to the gummy matrix and mixing the gummy matrix until the gelatin is completely dissolved;
maintaining the resulting gummy matrix at a constant temperature;
slow boiling the gummy matrix;
adding a sweetener to the gummy matrix;
incorporating ergogenic ingredients into the gummy matrix;
homogenously mixing the gummy matrix; and
molding the resulting gummy matrix.

13. The method of claim 12, wherein the gummy matrix is a mixed gel.

14. The method of claim 13, wherein the mixed gel comprises from 7 to 9 percent by weight gelatin.

15. The method of claim 12, wherein sugar is added to the gummy matrix from 5.0 to 8.0 percent by weight of the gummy matrix.

16. The method of claim 12, wherein a protein source is added to the gummy matrix.

17. The method of claim 16, wherein the protein source comprises from 14.0 to 22.0 percent by weight of the gummy matrix.

18. The method of claim 12, wherein gelatin is not added to the gummy matrix the matrix is at a temperature of from 55 to 70 degrees Celsius.

19. The method of claim 12, wherein ergogenic ingredients are only added after the gummy matrix has cooled to a temperature within the range of 40 to 45 degrees Celsius.
